(19) United States
(12) Patent Application Publication
(10) Pub. No.: US 2007/0155664 A1
(43) Pub. Date: Jul. 5, 2007

(54) Parathyroid hormone (pth) containing pharmaceutical compositions for oral use

(75) Inventors: Lisbeth Bonlokke Ranklove, Frederiksberg C (DK); Karin L. Christensen, Frederiksberg (DK); Jimmy H. Schlyter, Greve (DK); Hanne A. Moesgaard, Praesto (DK)

Correspondence Address: EDWARDS ANGELL PALMER & DODGE LLP, P.O. BOX 55874, BOSTON, MA, 02205, US

(73) Assignee: Nycomed Danmark A/S, Frederiksberg C (DK)

(21) Appl. No.: 10/561,876

(22) PCT Filed: Jul. 5, 2004
(86) PCT No.: PCT/DK04/00482

§ 371 Date: Feb. 6, 2007

(30) Foreign Application Priority Data
Jul. 4, 2003 (DK) PA 2003 01035

(51) Int. Cl. A61K 38/29

(52) U.S. Cl. 514/12

(57) ABSTRACT

A pharmaceutical composition for oral administration comprising PTH, wherein the in vitro release of PTH-when tested in a dissolution test of pharmacopoeia standard-is delayed with at least 2 hours and once the release starts, at least 90% w/w such as, e.g., at least 95% or at least 99% of all PTH contained in the composition is released within at the most 2 hours. The composition may also comprises a calcium containing compound and/or a vitamin, D. In particular, PTH is administered in combination with a calcium-containing compound for the treatment or prevention of bone-related diseases, so that I) an effective amount of a calcium-containing compound is administered to lower the plasma level of endogenous PTH, and II) an effective amount of PTH is administered to obtain a peak concentration of Pm once the endogeneous PTH level is lowered. This present a potential therapeutic or prophylactic regimen for bone-related disorders including osteoporosis.

### FIELD OF THE INVENTION

The present invention relates to pharmaceutical compositions containing a parathyroid hormone (PTH) optionally in combination with a suitable calcium and/or vitamin D containing compound for use in the prevention and/or treatment of conditions where a bone resorption inhibitor is indicated including subjects suffering from or at risk of e.g. osteoporosis.

Furthermore, the present invention relates to a novel pharmaceutical composition especially suitable for delivering proteins/peptides like PTH to specific parts of the gastrointestinal tract such as, e.g., the small intestine or the colon. The pharmaceutical composition is designed so that the release of the active substance is delayed by a combination of two principles, namely by combination of a pH controlled and/or a time controlled mechanism. Furthermore, after the release delay, the pharmaceutical composition is designed to release the active substance relatively fast to ensure that the active substance is ready for absorption via the GI mucosa in the small intestines and/or the colon.

### BACKGROUND OF THE INVENTION

Over the last decades an increasing number of peptides and proteins have been made available as therapeutic agents. Unfortunately, the full potential of these macromolecules has not been recognized because they normally require administration by injection. Studies done in animals and humans with PTH, PTH related peptides or analogs have demonstrated its usefulness in increasing bone formation and bone resorption and have prompted interest in its use for the treatment of osteoporosis and related bone disorders. In fact, Teriparatide, a recombinant parathyroid hormone (1-34) has been approved by the Food and Drug Administration (FDA) for parenteral treatment of osteoporosis in postmenopausal woman and in men with idiopathic or hypogonadal osteoporosis, who are at high risk for fracture.

However, oral administration and delivery of peptides like PTH and derivatives and analogs thereof represent a major challenge for oral delivery simply because the gastrointestinal tract is designed for the digestion of proteins or peptides from the meal, i.e. the conditions prevailing in the gastrointestinal tract degrade proteins and peptides and thus prevent the proteins and peptides from being absorbed as intact proteins and peptides.

WO 02/098453 (Novartis-Erfingungen Verwaltungsgesellschaft M.B.H.) relates to a method for orally administering parathyroid hormone (PTH) and salmon calcitonin. 5-CNAS is used as an absorption enhancer. The compositions employed are in the form of capsules only containing the substances to be tested, i.e. no pharmaceutically acceptable excipients are used.

WO 03/015822 (Novartis AG) relates to 5-CNAC as oral delivery agent for human parathyroid hormone fragment (hPTH). 5-CNAC is described to enhance the absorption of PTH after oral administration. The compositions administered are in the form of capsules consisting solely of hPTH or solely of a combination of hPTH and 5-CNAC, i.e. no pharmaceutically acceptable excipients have been used.

However, such compositions are not suitable for large scale production and accordingly, there is a need to develop pharmaceutical compositions that easily can be produced in large batch size and that are suitable for oral administration of peptides like PTH optionally in combination with other therapeutically and/or prophylactically active agents. To this end, an example of a suitable combination is a combination of PTH and a calcium salt. Recent studies have shown that a balanced dosage of PTH and adjunct intake of calcium and/or vitamin D, respectively, has a positive effect on decreasing bone degradation processes including osteoporosis.

During the last decades it has emerged that some active substances are subject to colon absorption. Thus, research and development have aimed at developing suitable delivery systems for targeting active substances to the colon. To this end a number of formulations have been suggested such as, e.g., a so-called time-controlled explosion system (TES) developed by Fujisawa (see e.g. EP-B-0210 540). Kinget et al. in J. Drug Targeting 1998, 6, 129-149, Leopold in PSTI 1999, 2, 197-204 and Bussemer et al. in Critical Review in Therapeutic Drug Carrier Systems 2001, 18, 433455 have given reviews on dosage forms for colon-specific drug delivery.

However, the known delivery systems for colon delivery result in relatively slow release of the active substance after a certain lag time. Such systems are therefore not particularly suitable in situations where it is desired to have a relatively fast release of the active substance in the colon. A relatively fast release of the active substance in the colon is especially of an advantage in those cases where the active substance is only absorbed in the ascending part of the colon or is sensitive to proteolytic activity or is poorly soluble and therefore requires a substantial amount of water/fluid to dissolve before absorption. Another situation is when the effect of the active substance is limited to a certain time period or when the absorption from the colon is poorer that from the small intestine.

Furthermore, the absorption of some active substances takes place in a specific part of the small intestine, i.e. they have a very narrow absorption window. For such substances it is also an advantage to develop a delivery system from which a fast release of the active substance takes place at a predetermined time corresponding to the time it takes to reach the specific part of the gastrointestinal tract that enables absorption of the active substance.

### DESCRIPTION OF THE INVENTION

The present invention relates to a pharmaceutical composition for oral administration comprising PTH, wherein the in vitro release of PTH—when tested in a dissolution test of pharmacopoeia standard—is delayed with at least 2 hours and once the release starts, at least 90% w/w such as, e.g., at least 95% or at least 99% of all PTH contained in the composition is released within at the most 2hours.

Such a composition is suitable for use in the treatment of a number of bone-related diseases. In particular administration thereof together with a calcium-containing compound in such a manner that the effect from calcium is fast whereas the effect from PTH is delayed, is believed to present a suitable therapeutic regimen. Accordingly, in an specific aspect, the invention relates to the use of a parathyroid hormone (PTH) in combination with a calcium-containing compound for the manufacture of a medicament for the treatment or prevention of bone-related diseases, wherein

i) an effective amount of a calcium-containing compound is administered to lower the plasma level of endogenous PTH,

ii) an effective amount of PTH is administered to obtain a peak concentration of PTH once the endogeneous PTH level is lowered.

In the following is given a description of the most important therapeutically and/or prophylactically active substance for use in a composition according to the invention. These active substances are parathyroid hormone (PTH), a calcium and/or vitamin D containing compound. Especially suitable is a combination comprising PTH and a calcium containing compound intended to be administered substantially simultaneous and designed to release calcium in the stomach (to achieve a relatively fast onset of action, namely to lower the plasma level of endogenous PTH) followed by a delay in release of PTH in the small intestine or in the colon (to enable absorption of PTH and to postpone the fast onset of action until the calcium containing compound has exerted its lowering effect on the endogenous PTH plasma level). In this manner the most beneficial therapy is envisaged.

Parathyroid Hormone

Parathyroid hormone (PTH) is a polypeptide consisting of 84 amino acids synthesized and secreted by the parathyroid glands. PTH can as a bone-building/anabolic agent is used alone or in combination with other current available osteoporosis drugs, which primarily prevent further bone loss.

A composition according to the present invention comprises a PTH, a fragment, an analog or a derivative thereof (in the present context the term PTH is used in a broad sense unless otherwise indicated, i.e. it includes fragments, analogs, derivatives, modifications such as PTH (full length, 1-84) or fragments thereof wherein one or more amino acid has been substituted by other amino acids or wherein one or more amino acid has been modified or deleted). As appears from the following, a composition according to the invention may apart from PTH contain other active substances such as, e.g. calcium or calcium and/or vitamin D containing compounds, vitamin D such as, e.g., vitamin D₃, other active substances suitable for use in the treatment of bone degradation processes, or combinations thereof. A suitable selection of active substances for use in a composition according to the invention is given herein. In a preferred embodiment, the composition comprises PTH or it comprises PTH and a calcium and/or vitamin D containing compound.

Fluoride, prostaglandin E₂ (PGE₂) and parathyroid hormone (PTH) are compounds, which have been shown to stimulate an increase in bone mass in humans or experimental animals. While fluoride can lead to an increase in fracture rates and PGE₂ may have unwanted side-effects, the actions of PTH seem to be relatively specific for bone. PTH or its amino-terminal (1-34) fragment increases bone mass in osteoporotic humans, normal rats and dogs. PTH improves bone loss in oestrogen-depleted rats in both a bone losing phase and with established osteopenia (Morietet al. Curr Pharm Des 2001; 7:671-87).

PTH is a principal regulator of calcium homeostasis through actions on kidney, intestine and bone. In kidney, PTH acts on cells within the distal tubular portion of the nephron to enhance calcium re-absorption at cortical sites and to block sodium, phosphate and bicarbonate re-absorption in the proximal tubule. The hormone also stimulates cells of the proximal tubule to produce 1,25-dihydroxy vitamin D₃ by enhancing 1-hydroxylase activity. It is this potent vitamin D metabolite, which then promotes calcium uptake from the diet in the intestinal mucosa.

The direct responsiveness of different tissues and organs to PTH is mediated by cell surface membrane receptors that are linked to the intracellular production of cyclic adenosine monophosphate (cAMP) and diacylglycerol-like cells in bone and in kidney and vascular smooth muscle cells. These receptors respond to full-length PTH (1-84) or its amino terminal fragment, PTH (e.g. PTH 1-34 etc), but not to mid-region or carboxy-terminal fragments. N-terminal fragments sometimes differ in activity from the native hormone, however, and the C-terminal region of PTH, acting through a receptor different from the classical PTH-1 receptor, initiates a variety of distinct biological activities. In particular, the C-terminal region of PTH, by promoting bone-cell apoptosis, may be important in opposing the anti-apoptotic effects of teriparatide in these cells, thereby maintaining normal bone-cell turnover. (Fox J. Curr Opin Pharmacol. 2002 Jun;2(3):338-44.) It is believed that the biological responses to administration of PTH (1-84) is similar to those observed with the more intensively studied amino-terminal PTH (1-34) or (1-38).

In bone, the mechanism of action of the hormone is much more complex. The acute response to endogenous PTH secretion is the net liberation of calcium and this is also true during continuous PTH secretion to the hormone at pharmacological levels. When PTH or any of its N-terminal (e.g. PTH 1-34) fragments are administered at supra-physiological levels in a pulsatile or intermittent (e.g. daily) fashion, however, the long-term effect is an up-regulation of bone formation that results in the net accumulation of newly mineralized bone tissue.

PTH can induce both bone resorption and bone formation and thus increase bone turnover. PTH usually exerts its action on bone to release calcium into the extracellular fluid as a process of bone remodeling and also to maintain the serum calcium concentration, but the exact mechanisms are not fully understood. In some circumstances PTH may exert actions on bone and can stimulate osteoblast proliferation and osteoblast function. The net effect of exogenous PTH administration on bone turnover depends on the pattern of PTH delivery; thus, a continuous infusion reduces bone volume whereas daily single injections result in a net increase.

In normal settings, 70-95% of circulating PTH is present as inactive C-terminal fragments. Intact PTH (1-84) constitutes only 5-30% of the circulating forms of the molecule. The biologically active N-terminal fragment is rapidly degraded in situ and there is little evidence that it is ever present in appreciable quantities in the circulation. Endogenous human PTH is rapidly metabolized primarily in the liver (60-70%) and kidney (20-30%).

Parathyroid hormone (PTH), especially intact human PTH (hPTH (1-84) and its various fragments hPTH (1-31), (1-34), (1-36), (1-38) and their modifications has been investigated for the use in the treatment of osteoporosis over the passed decades. In the present context PTH encompasses but is not limited to PTH, PTH analogues, PTH derivatives and substances that have a PTH activity or related activity. It has been found that human parathyroid hormone fragments, in which the C-terminal amino acid is amino acid 35 to 38, preferably 37 or 38 and at least the first N-terminal amino acid has been removed, and analogs and derivatives thereof stimulate osteoblast activity and maximize bone formation without undesirable levels of bone resorption, antibody formation, or tachyphylaxis. The human parathyroid hormone fragments can be represented in accordance with standard nomenclature by the formula (m-n) PTH: (3-38 PTH)-(28-38 PTH), (3-37 PTH) -(28-37 PTH), (2-35 PTH)-(2-38 PTH), and C-terminal amide derivative of the above mentioned where PTH is human parathyroid hormone (hPTH) or a pharmaceutically acceptable salt or hydrolysable ester thereof.

Of the various kind of anabolic agents tested in the treatment of osteoporosis intermittent injections of PTH has proven to be the most effective up to date (Seeman E. et al. Trends Endocrinol Metab 2001; 12 (7): 281-3). However, other administration routes may also be efficient.

Although chronic continuous excess of PTH markedly increases bone resorption, as seen in the typical example of primary hyperparathyroidism and osteitis fibrosa generalisata, intermittent PTH administration has been found to stimulate bone formation in animals, providing a basis for the use of PTH as a therapeutic agent for osteoporosis. In addition to dramatically increasing trabecular bone density and also sustaining cortical bone density, PTH administration increases bone strength and reduces the fracture rate, e.g. 40 μg/daily (1-34 PTH) (Neer et al, N Engl J Med 2001,10;344(19):1434-41). Administration of PTH in combination with antiresorptive agents such as oestrogen, calcitonine, vitamin D and bisphoshonates augments its effect e.g. 50, 75 or 100 μg/daily (1-84 PTH) for one year follow by 10 mg alendronate daily for one year (Rittmaster R S et al. J Clin Endo Met 2000,85:2129-2134).

It is generally believed that all patients should always be supplemented with calcium and/or vitamin D, e.g., 1000-1500 mg calcium and at least 400-800 IU vitamin D.

Because of its bone anabolic action, PTH is expected to be effective for osteoporosis in those of advanced age with suppressed bone remodelling, which might not respond favourably to antiresorptive agents. (Fujita T, BioDrugs 2001;15(11):721-8).

To sum up it seems that in order to enable a therapeutic effect of PTH it seems to be of utmost importance that the plasma concentration of PTH fluctuates or reaches a peak concentration. Moreover, it seems to be important that a high peak PTH plasma concentration rapidly decreases to a suitable low level. This is important in order to avoid or reduce the unwanted effect of an continuously increased PTH plasma level, which is release of calcium from the bone mass leading to a decreased BMD (bone mineral density), which in turn increases the risk for fracture and osteoporosis. In other words, treatment with a PTH requires due consideration to the positive as well as the negative effects of PTH. Such a treatment is advantageous supplemented with administration of calcium and/or vitamin D containing compound, which beneficial lower the effect on the plasma PTH between the one daily administrate of PTH in order to counteract the negative effects of endogenous PTH on calcium depletion from the bone mass.

To this end, the present inventors have developed a pharmaceutical composition comprising a PTH, wherein the PTH after oral administration is released after a certain period of time and moreover, PTH is released over a narrow time period in order to enable a sufficient therapeutic response. The lag time for release of PTH is designed in order to avoid any (or any significant) release in the stomach.

In the stomach, the protein/peptide will usually be very unstable under the strong acidic conditions in the stomach. Strong acidic conditions will favour hydrolysis, aggregation, and/or denaturation of the protein molecule, which usually will result in loss of biological activity.

If the orally administered protein/peptide reaches the small intestine, it will usually be digested by proteolytic enzymes, which are abundant in the region of the GI tract, both secreted (trypsin, chymotrypsin, elastase, carboxypeptidase A and B) and membrane bound (endopeptidase, amino-peptidase and carboxy-peptidase). If the enzymes do not digest the protein/peptide, the next barrier is the absorption through the epithelial cells in the small intestine. The “typical” globular protein molecules have a hydrophilic outer surface and a hydrophobic core. Combined with the large molecular weight a protein/peptide is not designed for the transcellular delivery, however possibly through the paracellular route. However molecular size is the major limitation for this route.

However, if the orally administered protein/peptide reaches the colon, it is expected to be less exposed to proteolytic enzymes, due to the less abundance of these enzymes in the colon. Absorption through the epithelial cells follows the same principles as mentioned above (small intestine). The content of the colon (the chyme) has a higher viscosity than in the other parts of the gastrointestinal tract, thus the mobility and diffusion of the large protein molecules to the epithelial membrane might be slowed down.

The present invention primarily aims at avoiding any (or substantially any) release of PTH in the stomach. The problems relating to degradation of PTH in the small intestine and in the colon are possible to overcome by employing suitable formulation technologies. Normally, however, it may pose a problem to delay the release within the gastrointestinal tract and at the same time ensure that the release, when it starts, is very fast as a delay in release gives often a more prolonged release.

Typical strategies to overcome the obstacles mentioned above relating to the conditions in the gastrointestinal tract that negatively influence the uptake of intact protein/peptides, are:

A: Protection against enzyme digestion:

A1: Ad enzyme inhibitors, which will slow down the digestion of the target protein and thus increase the chances of absorption. NB: Digestion of other proteins is slowed down as well.

A2: Change the protein molecule or ad some d-amino acids, or non-natural amino acids or derivatives.

A3: To encapsulate or protect the proteins by the design of the formulation (e.g. particulate systems)

B: Enhancement of the absorption

B1: Hydrofobization of the protein by lipid side chains (conjugated) or replacement of hydrophilic amino acids with more hydrophobic amino acids.

B2: Formulation design, e.g. emulsions, particle systems, muco-adhesive systems.

A composition according to the invention is designed so that release of PTH is primarily avoided in the stomach. Different types of compositions are described in the following based on the GI (gastrointestinal) target for release. Accordingly, in the following compositions are described, which are designed to be released in i) the small intestines (upper or lower part) or in ii) the colon. As it appears from the description below, different strategies are applied dependent on the GI target for release.

PTH dose: Normal therapeutic dose of PTH (1-84) is about 0.1 mg/dose, assuming bioavailability of the oral formulation of 1-5% the corresponding oral dose would be about 10-50 mg.

PTH and a Calcium and/or Vitamin D Containing Compound

As mentioned above, the present invention also provides a pharmaceutical composition comprising a PTH in combination with a therapeutically and/or prophylactically active calcium and/or vitamin D containing compound. Thus, in a specific embodiment the invention provides a pharmaceutical composition that contains a PTH together with another therapeutically active substance for use in the treatment of bone diseases. In a preferred aspect, such a substance is a calcium and/or vitamin D containing compound such as, e.g., a calcium salt and e.g. cholecalciferol. In this respect, such a combination is a formulation challenge, as PTH should not be released in the stomach (due to degradation in the stomach), whereas the calcium and/or vitamin D containing compound must be subjected to the acidic environment prevailing in the stomach in order to enable the desired absorption and therapeutic effect. However, as shown in the examples herein the present inventors have designed compositions enabling such a difference in release of two active substances.

Calcium and/or Vitamin D Containing Compound

Calcium and/or vitamin D containing compound contained in a composition according to the invention is a physiologically tolerable calcium and/or vitamin D containing compound that is therapeutically and/or prophylactically active.

Calcium is essential for a number of key functions in the body, both as ionized calcium and a calcium complex (Campell A K. Clin Sci 1987; 72:1-10). Cell behaviour and growth are regulated by calcium. In association with troponin, calcium controls muscle contraction and relaxation (Ebashi S. Proc R Soc Lond 1980; 207:259-86).

Calcium selected channels are a universal feature of the cell membrane and the electrical activity of nerve tissue and the discharge of neurosecretory granules are a function of the balance between intracellular and extra cellular calcium levels (Burgoyne R D. Biochim Biophys Acta 1984;779:201-16). The secretion of hormones and the activity of key enzymes and proteins are dependent on calcium. Finally calcium as a calcium phosphate complex confers rigidity and strength on the skeleton (Boskey A L. Springer, 1988:171-26). Because bone contains over 99% of the total body calcium, skeletal calcium also serves as the major long-term calcium reservoir.

Calcium salts such as, e.g., calcium carbonate is used as a source of calcium especially for patients suffering from or at risk of osteoporosis. Moreover, calcium carbonate is used as an acid-neutralizing agent in antacid tablets.

As mentioned above, calcium has a number of important functions within the mammalian body in particular in humans. Furthermore, in many animal models, chronic low calcium intake produces osteopenia. The osteopenia affects cancellous bone more than cortical bone and may not be completely reversible with calcium supplementation. If the animal is growing reduced calcium intake leads to stunting. In the premature human neonate, the higher the calcium intake, the greater the intake, the greater the increase in skeletal calcium accretion which, if high enough, can equal gestational calcium retention. During growth chronic calcium deficiency causes rickets. Calcium supplements in both pre- and postpubertal healthy children leads to increased bone mass. In adolescents, the higher the calcium intake, the greater the calcium retention, with the highest retention occurring just after menarche. Taken together, these data suggest that in children and adolescents, considered to be taking an adequate intake of calcium, peak bone mass can be optimized by supplementing the diet with calcium. The mechanisms involved in optimizing deposition of calcium in the skeleton during growth are unknown. They are probably innate properties of the mineralization process that ensures optimal calcification of the osteoid if calcium supplies are high. The factors responsible for stunting of growth in states of calcium deficiency are also unknown but clearly involve growth factors regulating skeletal size.

In adults calcium supplementation reduces the rate of age-related bone loss (Dawson-Hughes B. Am J Clin Nut 1991 ;54:S274-80). Calcium supplements are important for individuals who cannot or will not achieve optimal calcium intakes from food. Furthermore, calcium supplement is important in the prevention and treatment of osteoporosis etc.

A review of 20 prospective calcium trials in postmenopausal women concludes that calcium supplementation on reduced bone loss is on average by about 1% year. In the elderly, calcium supplementation also reduces bone loss and the lower the prevailing dietary intake, the better the response in bone. The effect of calcium intake on the skeleton is to reduce the number of osteoporotic fractures, although this effect is not consistent across studies (Cumming R G et al. J Bone Miner Res 1997; 12:1321-9). The mechanism by which calcium supplementation slows bone loss is probably through a reduction in serum PTH. With age there is an increase in serum PTH and bone turnover due to the combined effects of reduced calcium intake and absorption and to vitamin D insufficiency. Calcium supplementation is most effective in this situation. Where PTH is already suppressed, such as in immobilisation and acute oestrogen deficiency, calcium supplementation is less likely to be so effective.

Furthermore, calcium may have anticancer actions within the colon. Several preliminary studies have shown high calcium diets or intake of calcium supplementation is associated with reduced colon rectal cancer. There is increasing evidence that calcium in combination with acetylsalicylic acid (ASA) and other non-steroidal anti-inflammatory drugs (NSAIDS) reduce the risk the risk of colorectal cancer.

Recent research studies suggest that calcium might relieve premenstrual syndrome (PMS). Some researchers believe that disruptions in calcium regulation are an underlying factor in the development of PMS symptoms. In one study, half the women of a 466 person group of pre-menopausal women from across the U.S. were tracked for three menstrual cycles and were given 1200 mg of calcium supplements daily throughout the cycle. The final results showed that 48% of the women who took placebo had PMS related symptoms. Only 30% of those receiving calcium tablets did.

Calcium Kinetics

The calcium content of the westemised diet is about 1 g/day. However, dietary calcium is present in only a few calcium-rich foods and the range in calcium intake, both within and between individuals is wide. In humans, absorption of calcium largely from the duodenum-jejunum is intermittent with meals. Calcium loss from the gut as endogenous secretions is passive and amounts to about 100 mg/day. On the other hand, calcium is absorbed by both active and passive mechanisms (Miller J. Z. et al. Am Inst Nutr 1990:265-74). On average, absorption in the young adult is only about 30% efficient. The main regulator of calcium absorption efficiency in humans is serum 1,25(OH)₂ vitamin D concentration, and although absorption efficiency increases as calcium intake decreases, it never achieves 100% efficiency. Once absorbed, the major flow of calcium is to bone and to kidney. In the kidney about 98% of the calcium that is filtered each day is reabsorbed, mainly under the regulation of parathyroid hormone (PTH) concentrations. The 2% un-reabsorbed calcium appears in urine as an obligatory calcium loss. In the bone of young adults, about 500 mg/day of calcium is deposited at the formation surfaces by osteoblasts and a similar amount released back to serum at the resorption surfaces by osteoclasts (Newton-John H et al. Clin Orthop 1970; 71:229-52). The overall result is that the skeleton remains in mineral balance. In older adults, however, age-related loss of bone occurs and there is a universal net loss of calcium from the skeleton. In children the rates of calcium transport are two to three times higher than the young adults, with formation greater than resorption such that there is net retention of calcium of about 300 mg/day and gain in bone (Wastney M E et al. Am J Physiol 1996; 271:208-16).

Gastric acidity assists in dissolving components of a standard meal. All calcium salts are more soluble in acidic media. Calcium carbonate and calcium phosphate are relatively water-insoluble and therefore clinical research support that calcium absorption from these salts is dependent on gastric acid production.

Calcium Homeostasis

In humans, the normal range of total calcium in serum is maintained at 8.8-10.2 mg/100 ml i.e. within about 15% of the mean concentration. About 40% of this calcium is bound to protein, 10% is complex bound with phosphate, sulphate and citrate and the remaining 50% is present as ionic calcium. The concentration of ionized calcium in serum is closely regulated through negative feedback of calcium on the secretion of PTH from the parathyroid glands and the secretion of 1,25 (OH)₂ vitamin D from the kidney. In the parathyroid gland, the reduction of PTH secretion in response to a rise in serum calcium is dependant on the integrity of a calcium- sensing receptor. In the kidney, change in PTH-secretion is the major regulator of 1,25 (OH)₂ vitamin D production although serum calcium and serum phosphate also affect production. In addition, serum 1,25 (OH)₂ vitamin D also plays a major role in this homeostatic mechanism by regulating PTH secretion and its own production and catabolism.

A calcium containing compound for use according to the invention may be e.g. bisglycino calcium, calcium acetate, calcium carbonate, calcium chloride, calcium citrate, calcium citrate malate, calcium comate, calcium fluoride, calcium glubionate, calcium gluconate, calcium glycerophosphate, calcium hydrogen phosphate, calcium hydroxyapatite, calcium lactate, calcium lactobionate, calcium lactogluconate, calcium phosphate, calcium pidolate, calcium stearate and tricalcium phosphate. Other calcium sources may be water-soluble calcium salts, or complexes like e.g. calcium alginate, calcium-EDTA and the like or organic compounds containing calcium like e.g. calcium organophosphates. Use of bone meal, dolomite and other unrefined calcium sources is discouraged because these sources may contain lead and other toxic contaminants. However, such sources may be relevant if they are purified to a desired degree.

Of specific interest is bisglycino calcium, calcium acetate, calcium carbonate, calcium chloride, calcium citrate, calcium citrate malate, calcium cornate, calcium fluoride, calcium glubionate, calcium gluconate, calcium glycerophosphate, calcium hydrogen phosphate, calcium hydroxyapatite, calcium lactate, calcium lactobionate, calcium lactogluconate, calcium phosphate, calcium pidolate, calcium stearate and tricalcium phosphate. Mixtures of different calcium containing compound may also be used. As appears from the examples herein, calcium carbonate is especially suitable for use as calcium containing compound and calcium carbonate has a high content of calcium.

Calcium is absorbed actively in the duodenum and the jejunum and passively in the ileum; only 20-33% of the oral administered dose is absorbed. Normally, a composition according to the invention contains an amount of the calcium containing compound corresponding to from about 100 to about 1000 mg Ca such as, e.g., from about 150 to about 800 mg, from about 200 to about 700 mg, from about 200 to about 600 mg or from about 200 to about 500 mg Ca.

Normally, the dose of calcium for therapeutic or prophylactic purposes is from about 350 mg (e.g. newborn) to about 1200 mg (lactating women) daily. The amount of the calcium in the tablets can be adjusted to that the tablets are suitable for administration 1-4 times daily, preferably once or twice daily.

Vitamin D

Function

In addition to its action on calcium and skeletal homeostasis, vitamin D is involved in the regulation of several major systems in the body. The actions of vitamin D are medicated at the genome by a complex formed by 1,25—(OH)₂vitamin D mainly produced in the kidney, with the vitamin D receptor (VDR). The latter is widely distributed in many cell types. The 1,25—(OH)₂vitamin DNDR complex has important regulatory roles in cell differentiation and in the immune system. Some of these actions are probably dependant on the ability of certain tissues other than the kidney to produce 1,25—(OH)₂vitamin D locally and act as a paracrine (Adams J S et al. Endocrinology 1996; 137:4514-7).

Metabolism

The major source of vitamin D is the skin where it is produced by the action of ultraviolet light on steroid precursors. Vitamin D, like calcium, is also present in a limited number of foods but although dietary sources can be important under circumstances of decreased sunlight exposure, vitamin D is not a true vitamin. It is a pro-steroid hormone that is biologically inert until metabolized (Block G. Am J Epidemiol 1985; 122:13-26). In the liver, vitamin D is metabolized to 25—OH vitamin D, which functions as the major storage form by virtue of its long half-life due to high affinity for the vitamin D binding protein (DBP) in blood. In the kidney 25—OH vitamin D is further metabolized by a 1α-hydroxylase enzyme to 1,25—(OH)₂vitamin D, the hormone responsible for the biological effects of vitamin D. The activity of the 1α-hydroxylase enzyme is tightly controlled by the blood levels of PTH, calcium and phosphate and by 1,25—(OH)₂vitamin D itself. Because serum 1,25—(OH)₂vitamin D has a much higher affinity for the VDR and a mush lower affinity for DBP than 25—OH vitamin D, 1 ,25—(OH) ₂ vitamin D is responsible for the action of vitamin D except under circumstances of pharmacological concentrations of 25—OH vitamin D in serum. These occur with oral consumption of either vitamin D or 25—OH vitamin D and lead to vitamin D intoxication (Monkawa T et al. Bioche Biophy Res Commu 1997; 239;527-33).

Skeletal Pathophysiology

In humans, deficiency of vitamin D results in rickets in children and osteomalacia in adults. The basic abnormality is a delay in the rate of mineralization off osteoid as it is laid down by the osteoblast (Peacock M. London Livingstone, 1993:83-118). It is not clear whether this delay is due to a failure of a 1 ,25—(OH)₂ vitamin D-dependant mechanism in the osteoblast or to reduced supplies of calcium and phosphate secondary to malabsorption or a combination of both. Accompanying the mineralization delay, there is reduced supply of calcium and phosphate, severe secondary hyperparathyroidism with hypocalcaemia and hypophosphatemia and increased bone turnover.

Vitamin D insufficiency, the preclinical phase of vitamin D deficiency, also causes a reduced calcium supply and secondary hyperparathyroidism, albeit of a milder degree than found with deficiency. If this state remains chronic, osteopenia results. The biochemical process underlying this state of calcium insufficiency is probably inappropriate levels of 1,25—(OH)₂ vitamin D due to a reduction in its substrate 25—OHD (Francis R M et al. Eur J Clin Invest 1983; 13:391-6). The state of vitamin D insufficiency is most commonly found in the elderly. With age there is a decrease in serum 25—OH vitamin D due to decreased sunlight exposure and possible to decreased skin synthesis. Furthermore, in the elderly the condition is exacerbated by a decrease in calcium intake and a paradoxical decrease in calcium absorption. The reduction in renal function with age giving rise to reduced renal 1,25—(OH)₂ vitamin D production may be a contributing factor. There are a number of studies of the effects of vitamin D supplementation on bone loss in the elderly. Some are without calcium supplementation and others are with calcium supplementation. It appears from the studies that although vitamin D supplementation is necessary to reverse deficiency and insufficiency, it is even more important as far as the skeleton is concerned to provide calcium supplementation since the major skeletal defect is calcium deficiency. In literature based on clinical trials, recent findings suggest trends of need for higher doses of vitamin D for the elderly patients (Compston J E. BMJ 1998;317:1466-67). An open quasi-randomised study of annual injections of 150.000-300.000 IU of vitamin D (corresponding to approx. 400-800 lU/day) showed a significant reduction in overall fracture rate but not in the rate of hip fracture in treated patients (Heikinheimo R J et al. Calcif Tissue Int 1992; 51:105-110). From a recently published trial was concluded that four monthly—four times/yearly supplementation with 100.000 IU oral vitamin D (corresponding to approx.800 IU/day) may prevent fractures, however does not decrease PTH adequately, suggesting that a more frequent dose may be considered in future trials.

One aspect of vitamin intoxication is increased bone resorption. Both 25—OH vitamin D and 1,25—(OH)₂vitamin D at high concentrations cause increased bone resorption in vitro and in vivo which can be blocked by antiresorptive agents such as estrogens and bisphoshonates (Gibbs et al. Postgrad Med J.1986;62:937-8). In the long term excess vitamin D leads to osteopenia (Adams et al. Annal Intem Med 1997:127; 203-6).

Recommended Daily Allowance (RDA) of Calcium and Vitamin D₃

(European Commission. Report on osteoporosis in the European Community. Action for prevention. Office for official Publications of the European Communities, Luxembourg 1998):

| | | |
|---|---|---|
| Group Age (years) | Calcium (mg)* | Vitamin D₃ (μg) |

| | | | |
|---|---|---|---|
| Newborn | 0-0.5 | 400 | 10-25 |
| | 0.5-1.0 | 360-400 | 10-25 |
| Children | 1.0-3.0 | 400-600 | 10 |
| | 4.0-7.0 | 450-600 | 0-10 |
| | 8.0-10 | 550-700 | 0-10 |
| Men | 11-17 | 900-1000 | 0-10 |
| | 18-24 | 900-1000 | 0-15 |
| | 25-65 | 700-800 | 0-10 |
| | 65+ | 700-800 | 10 |
| Women | 11-17 | 900-1000 | 0-15 |
| | 18-24 | 900-1000 | 0-10 |
| | 25-50 | 700-800 | 0-10 |
| | 51-65 | 800 | 0-10 |
| | 65+ | 700-800 | 10 |
| Pregnant | | 700-900 | 10 |
| Lactating | | 1200 | 10 |

*RDA of calcium varies from country to country and is being re-evaluated in many countries.

In general, the compositions according to the invention are based on the following targets in vivo and the corresponding target dissolution profiles in vitro.

In Vivo and In Vitro Targets for Release of PTH

In Vivo Targets in the Gastrointestinal Tract

The plasma profile for PTH depends on the particular PTH employed. Accordingly, the plasma profile for compositions comprising PTH 1-84 or PTH 1-34 should be the following:

Profile Wanted for PTH-84:

The ratio between the peak concentration and the basis concentration of PTH, i.e. Cₘₐₓ/C_{basis} is in a range from about 2 to about 20, such as from about 4 to about 18, from about 6 to about 17 or from about 8 to about 15 times.

Tₘₐₓ after the absorption begins, is about 1 hour (interval 0.5-2.5 hours), outer limits 0.2-6 hours.

W₅₀, i.e. the time period during which the concentration of PTH is 50% or more of the peak concentration, is in a range of from about 0.1 to about 6 hours such as, e.g., from about 1 to about 3 hours such as about 1 hour.

Profile Wanted for PTH 1-34:

The ratio between the peak concentration and the basis concentration of PTH, i.e. Cₘₐₓ/C_{bass} is in a range from about 1 to about 10 such as, e.g., from about 4 to about 9.

Tₘₐₓ after the absorption begins, is about 0.5 hour (interval 0.2-1 hours), outer limits 0.1-3 hours

W₅₀, i.e. the time period during which the concentration of PTH is 50% or more of the peak concentration, is in a range of from about 0.1-4 hours such as, e.g., from about 0.5 to about 1.5 hours).

Absorption of PTH

Absorption of PTH should start when released from the composition in the gastrointestinal tract. Due to different formulation technologies employed to provide compositions according to the present invention, it may be released in the small intestines or in colon.

Small Intestine

Compositions designed to release a PTH in the jejunum (i.e. the GI target is the jejunum) are designed to have a lag time (i.e. a time period after administration wherein release of PTH is substantially avoided) corresponding to approx 0.5-1.5 hours after gastric emptying upon which PTH is rapidly released. In a specific embodiment of the invention compositions designed to release PTH in the small intestine should have a relatively high load of PTH-stabilization agents (i.e. inhibitors that inhibits degradation of the PTH in this part of the small intestine), and all kinds of different absorption enhancers may be employed.

Compositions designed to release a PTH in the ileum (i.e. the GI target is the ileum) are designed to have a lag time corresponding to approx. 2-4 hours after gastric emptying upon which PTH is rapidly released. In a specific embodiment of the invention such compositions contain a suitable load of PTH-stabilization agents (inhibitors). Bile salts should be as used as absorption enhancers in order to take advantage of the natural absorption of bile salts in this area (due to enterohepatic recirculation of natural bile salts from the bile). This formulation strategy seems to be the very promising since the absorption enhancers (bile salts) situated in the jejunum will mimic a natural process. The use of other types of enhancers throughout the gastrointestinal tract might give an unwanted absorption of other ingested proteins and, accordingly, bile salts are preferred as absorption enhancers in composition for ileum delivery.

Colon

Compositions designed to release a PTH in the colon (i.e. the GI target is the colon) are designed to have a lag time corresponding to approx. 3-6 hours after gastric emptying, most likely 3-4 hours after gastric emptying, target timing after administration of formulation to the patient should be 5.5 hours. The inventors have found that it is important that such compositions contain a suitable load of PTH-stabilization agents (inhibitors) and different kind of absorption enhancers may also be included in such compositions.

All types of compositions according to the present invention (i.e. irrespective of the GI target for PTH release) are designed to avoid release in the stomach, e.g. by use of an enteric polymer. Moreover it is contemplated that the permeability of PTH (once released under the conditions prevailing or established in the specific part of the GI tract) through the GI mucosa to the systemic circulation is relatively fast e.g. due to the presence of an absorption enhancer. When the effects derived from the enhancer and the PTH-stabilizing agent are lost, PTH is expected to be degraded due to the normal conditions prevailing in the GI tract, i.e. no further absorption of intact PTH is expected. This issue is important in order to obtain a narrow peak (i.e. a fast rise followed by a fast decline in PTH plasma concentration) and to avoid a sustained or prolonged uptake of PTH that would lead to a plasma concentration level of PTH that is unwanted due to the negative effect on calcium depletion from the bone.

In Vitro Profiles—Dissolution

In principle all kinds of pharmaceutical compositions can be used, i.e. a composition according to the invention may be solid (e.g. tablets, capsules, sachets, powders, granules, beads, pellets etc.), semi-solid or in liquid form (including solutions, emulsions and suspensions). In particular with respect to delivery to the small intestine and/or the colon, a number of formulation technologies may be employed. One of these are more specifically described herein, but other technologies may equally well be applied including, but not limited to, emulsions (see e.g. Tarr-B D et. al, Pharm. Res. 1989; 6(1):40-3, hydrogels (see e.g. Lowmann. A M et. al., J. Pharm. Sci 1999; 88(9):933-7) and ((Rubinstein-A et. al. 1995; 41:291-5), microemulsions (see e.g. Watnasirichaikul-S et. al, Pharm. Res 2000; 17(6):684-9), particulate systems (see e.g. Carino-G P et. al, J. Control. Release 2000; 65(1-2):261-9, enzyme-controlled drug delivery, e.g. commercial coatings that are degradable by microbial enzymes in the colon like ethylcellulose with amylose, or e.g. capsules containing PTH (e.g. coated with a protective coating) dispersed in a suitable oil. The following types of formulation suitable for use according to the inventions are included for illustrative purposes and are not intended to limit the invention in any way. However, solid dosage forms such as e.g. tablets, pellets and capsules are dosage forms that have good patient acceptability and therefore, these types of formulations are used in the following to illustrate the general principles of the invention. The dissolution referred to below is determined according to pharmacopoeia standards using a suitable dissolution apparatus and dissolution conditions (media and temperature). A person skilled in the art knows how to choose a suitable method based on the particular composition and the GI release target. The dissolution tests described e.g. in USP/NF or Ph. Eur. are generally applicable in the present context. In some cases an enteric polymer may be employed in a composition of the present invention, which enteric polymer has a pH cut off (i.e. the lowest pH value by which the enteric polymer is soluble at a temperature of 37° C.) that is above pH 6. In such cases the following dissolution tests must be carried out using a buffer (after the initial testing at an acidic pH) having a pH value that simulates the in vivo conditions in the particular segments of the GI tract. A person skilled in the art will know how to adapt the dissolution conditions thereto. A specific example is given in Example 2 herein.

Pellets, tablets and capsules (all should be enteric coated):
Pellets for release in jejunum, ileum and colon
Tablets for release in jejunum
Capsules for release in jejunum

Jejunum Delivery (e.g. Tablets, Capsules or Pellets)

Dissolution at 0.1 N HCl (approx. pH 1.2) for 2 hours drug release approx. 0-1% w/w (limits 0-10% w/w),

change of pH to pH 6.8

dissolution at pH 6.8

time after start at pH 6.8

| | |
|---|---|
| at 15 min. | approx. 20% w/w (limits 0-50% w/w) |
| at 30 min. | approx. 80% w/w (limits 25-100% w/w) |
| at 60 min. | approx. 100% w/w (limits 50-100% w/w) |

More specifically:

Dissolution at a first pH value such as, e.g., 0.1 N HCl (approx. pH 1.2) for 2 hours at 37° C.: at the most about 10% w/w such as, e.g., not more than about 7.5% w/w such as, e.g., not more than about 5% w/w, not more than about 2.5% w/w or not more than about 1% w/w of PTH contained in the composition is released at the first pH value below about 4.0 (in specific embodiments of the invention, this first pH value is below about 3.5, such as, e.g., below about 3.0, below about 2.5, below about 2.0, below about 1.5 or a pH value corresponding to that of 0.1 N HCl); then change of pH to pH 6.8 and

dissolution at pH 6.8:

| | |
|---|---|
| at 15 min: | about 0-50% w/w such as, e.g., 0-40% w/w, 0-35% w/w, |
| | 0-30% w/w, 5-50% w/w, 5-40% w/w, 5-35% w/w, |
| | 5-30% w/w, 10-50% w/w, 10-40% w/w, 10-35% w/w |
| | or 10-30% w/w such as e.g. about 20% w/w, |
| at 30 min. | about 25-100% w/w such as, e.g., 25-95% w/w, 25-90% |
| | w/w, 25-85% w/w, 30-100% w/w, 30-95% w/w, 30-90% |
| | w/w, 30-85% w/w, 35-100% w/w, 35-95% w/w, 35-90% |
| | w/w, 35-85% w/w, 40-100% w/w, 40-95% w/w, 40-90% |
| | w/w, 40-85% w/w, 45-100% w/w, 45-95% w/w, 45-90% |
| | w/w, 45-85% w/w, 50-100% w/w, 50-95% w/w, 50-90% |
| | w/w, 50-85% w/w, 55-100% w/w, 55-95% w/w, 55-90% |
| | w/w, 55-85% w/w, 60-100% w/w, 60-95% w/w, 60-90% |
| | w/w, 60-85% w/w, 65-100% w/w, 65-95% w/w, 65-90% |
| | w/w, 65-85% w/w, 70-100% w/w, 70-95% w/w, 70-90% |
| | w/w, 70-85% w/w such as e.g. about 80% w/w, |
| at 60 min. | about 50-100% w/w such as, e.g., 50-95% w/w, 50-90% |
| | w/w, 50-85% w/w, 55-100% w/w, 55-95% w/w, 55-90% |
| | w/w, 55-85% w/w, 60-100% w/w, 60-95% w/w, 60-90% |
| | w/w, 60-85% w/w, 65-100% w/w, 65-95% w/w, 65-90% |
| | w/w, 65-85% w/w, 70-100% w/w, 70-95% w/w, 70-90% |
| | w/w, 70-85% w/w, 80-100% w/w, 80-95% w/w such as |
| | e.g. about 95-100% w/w |

Once a specific dissolution profile is decided that suitably corresponds to the desired in vivo release, the permitted variability in release at any given time period should not exceed a total numerical difference of ×10% (in the following denoted % point) such as, e.g., at the most about ±7.5% or at the most about ±5% of the labelled content of the active substance (see CPMP (Committee for proprietary medicinal products (EU)) Guideline made by EMEA (The European Agency for the Evaluation of Medicinal Products): “Note for Guidance on quality of modified release products: A: oral dosage forms. B: transdermal dosage forms, section I (quality)”, CPMP/QWP/604196, 29 Jul. 1999). The 10% point leads e.g. to a total variability of 20%: a requirement of 50+/−10% thus means an acceptance range from 40-60%.

Pellets

Ileum Delivery (Lag Time Approx. 2 Hours After Gastric Emptying)

Dissolution at 0.1 N HCl (approx. pH 1.2) for 2 hours drug release 0-1% w/w (limit 0-10% w/w),

change of pH to pH 6.8

dissolution at pH 6.8

time after start at pH 6.8

| | |
|---|---|
| at 2 hours 30 min | approx. 20% w/w (limits 0-50% w/w) |
| at 3 hours 30 min | approx. 80% w/w (limits 25-100% w/w) |
| at 4 hours 30 min. | approx. 100% w/w (limits 50-100% w/w) |

i.e. the test period is different from that mentioned above under jejunum delivery, but otherwise the same conditions and ranges stated above are also applicable for compositions according to the invention for ileum (as well as colon delivery cf. below) delivery.

Pellets

Colon Delivery (Lag Time Approx. 3.5 Hours After Gastric Emptying)

Dissolution at 0.1 N HCl (approx. pH 1.2) for 2 hours drug release approx. 0-1% W/W (limits 0-10% w/w),

change of pH to pH 6.8

dissolution at pH 6.8

time after start at pH 6.8

| | |
|---|---|
| at 4 hours | approx. 20% w/w (limits 0-50% w/w) |
| at 5 hours | approx. 80% w/w (limits 25-100% w/w) |
| at 6 hours | approx. 100% w/w (limits 50-100% w/w) |

i.e. the test period is different from that mentioned above under jejunum delivery, but otherwise the same conditions and ranges stated above are also applicable for compositions according to the invention for ileum as well as colon delivery.

As mentioned above, the lag time is from about 0.5 to about 8 hours. In specific embodiments, the lag time is from about 1.0 to about 7 hours such as, e.g., from about 1.5 to about 6 hours, from about 2.0 to about 5 hours or from about 2.5 to about 4.5 hours or from about 2.5 to about 4 hours. In those cases where the pharmaceutical composition is intended for delivering an active substance to the colon, the lag time is normally from about 2.5 to about 4.5 hours. However, as appears from the above, a pharmaceutical composition of the present invention is also suitable for use in those cases where the active substance is absorbed from a specific part of the small intestine. In such cases, the lag time is shorter than when colon absorption or delivery is the target.

An important feature of a pharmaceutical composition of the present invention is that the active substance is relatively fast released after the predetermined lag time. Furthermore, the pharmaceutical composition should be designed to release all or almost the whole content of active substance.

Accordingly, after the above-mentioned lag time—at least about 60% w/w such as, e.g., at least about 70% w/w, at least about 75% w/w, at least about 80% w/w, at least about 85% w/w, at least about 90% w/w, at least about 95% w/w or at least 99% w/w of the active substance contained in the composition is normally released within the second time period of not more than about 2 hours.

In specific embodiments, the said second time period is not more than about 90 min such as, e.g., not more than about 60 min, not more than about 50 min, not more than about 45 min, not more than about 40 min, not more than about 35 min, not more than about 30 min, not more than about 25 min, not more than about 20 min, not more than about 15 min, not more than about 10 min of not more than about 5 min. Normally the second time period is about 30-60 min.

Pharmaceutical Compositions Comprising PTH and a Calcium and/or Vitamin D Containing Compound

Such compositions may be in the form of a single composition containing the active substances, it may e.g. be in the form of pellets/granules containing different types of pellets/granules e.g. one containing PTH (pellets) and the other type (granules) containing the calcium and/or vitamin D containing compound (and the two types of pellets/granules may be contained in capsules, sachets or the like), or it may be in the form of a kit comprising two distinct component, one comprising PTH and the other comprising the calcium and/or vitamin D containing compound. Further components may also be included in any of the above-mentioned types of compositions, in particular a further component containing an additional dose of a calcium and/or vitamin D containing compound to be administered at another time than the combination of PTH and calcium.

In the following is described the in vivo and in vitro targets for pharmaceutical compositions according to the invention comprising a combination of PTH and a calcium and/or vitamin D containing compound.

In Vivo Situation

The compositions according to the invention is designed so that a plasma curve for PTH

i) first gives a lower plasma level compared with the basis line due to absorption of calcium and

ii) then a peak due to absorption of PTH from the composition.

The lowering of PTH plasma level as well as the peak PTH are believed to be beneficial for bone growth. The PTH from the composition should not be absorbed during the time period, where calcium gives the beneficial lowering effect on the plasma PTH. The effect of ingested calcium on plasma PTH seems to stop after about 4 hours, thus, PTH can be released form the formulation after 4 hours after administration. This means that we can combine a fast release of calcium with a delayed release (burst) of PTH with 4 hours or more apart.

Accordingly, a composition according to the invention comprising a combination of a PTH and a calcium and/or vitamin 0 containing compound is suitable for a GI target for release of PTH in the ileum or colon. Furthermore, it is very important that calcium is released in the stomach in order to subject calcium to the acid environment prevailing in the stomach.

Formulation Types

As mentioned above, all formulation technologies suitable for small intestine or colon delivery can be applied. An example may be PTH containing pellets for release in ileum and colon. The PTH part of the formulation should be enteric coated to avoid release in the stomach and time controlled to obtain the suitable lag time required to delay the release of PTH until it reaches the ileum or the colon, while calcium must not be enteric coated. Calcium can be given as a separate composition or as pellets/granules or as powder mixed with other inactive ingredients and the PTH containing pellets.

In Vitro Profiles—Dissolution

Disintegration

The composition (or, if two separate components are employed, at least the one comprising the calcium and/or vitamin D containing compound) should have a disintegration time of 15 min or less (normally within approx. 5-15 min.). A fast disintegration time ensures a fast release of the calcium and/or vitamin D containing compound.

Dissolution

Composition (e.g. Pellets) PTH+Calcium and/or Vitamin D Containing Compound Calcium Containing Compound Formulated With Either PTH Pellets for Ileum or Colon Delivery

Calcium Dissolution at 0.1 N HCl (pH approx. 1.2):

| | |
|---|---|
| at 15 min. | approx. 20% w/w (limits 0-50% w/w) |
| at 30 min. | approx. 80% w/w (limits 25-100% w/w) |
| at 45 min. | approx. 100% w/w (limits 50-100% w/w) |

More specifically:

Dissolution at 0.1 N HCl (approx. pH 1.2)

| | |
|---|---|
| at 15 min: | about 0-50% w/w such as, e.g., 0-40% w/w, 0-35% w/w, |
| | 0-30% w/w, 5-50% w/w, 5-40% w/w, 5-35% w/w, |
| | 5-30% w/w, 10-50% w/w, 10-40% w/w, 10-35% w/w |
| | or 10-30% w/w such as e.g. about 20% w/w, |
| at 30 min. | about 25-100% w/w such as, e.g., 25-95% w/w, 25-90% |
| | w/w, 25-85% w/w, 30-100% w/w, 30-95% w/w, 30-90% |
| | w/w, 30-85% w/w, 35-100% w/w, 35-95% w/w, 35-90% |
| | w/w, 35-85% w/w, 40-100% w/w, 40-95% w/w, 40-90% |
| | w/w, 40-85% w/w, 45-100% w/w, 45-95% w/w, 45-90% |
| | w/w, 45-85% w/w, 50-100% w/w, 50-95% w/w, 50-90% |
| | w/w, 50-85% w/w, 55-100% w/w, 55-95% w/w, 55-90% |
| | w/w, 55-85% w/w, 60-100% w/w, 60-95% w/w, 60-90% |
| | w/w, 60-85% w/w, 65-100% w/w, 65-95% w/w, 65-90% |
| | w/w, 65-85% w/w, 70-100% w/w, 70-95% w/w, 70-90% |
| | w/w, 70-85% w/w such as e.g. about 80% w/w, |
| at 60 min. | about 50-100% w/w such as, e.g., 50-95% w/w, 50-90% |
| | w/w, 50-85% w/w, 55-100% w/w, 55-95% w/w, 55-90% |
| | w/w, 55-85% w/w, 60-100% w/w, 60-95% w/w, 60-90% |
| | w/w, 60-85% w/w, 65-100% w/w, 65-95% w/w, 65-90% |
| | w/w, 65-85% w/w, 70-100% w/w, 70-95% w/w, 70-90% |
| | w/w, 70-85% w/w, 80-100% w/w, 80-95% w/w such as |
| | e.g. about 95-100% w/w |

PTH Containing Pellets for Ileum or Colon Delivery

The same dissolution patterns as mentioned herein before apply. Ileum delivery (lag time approx. 2 hours after gastric emptying) and colon delivery (lag time approx. 3.5 hours after gastric emptying)

Osteoporosis & Therapeutic Choices—Use of Compositions According to the Invention

Disease Background

Incidence

Osteoporosis is a generalized skeletal disorder in which bone loss and deterioration of bone micro-architecture reduce bone strength to the point that fractures may occur with minimal trauma. Osteoporosis is major public health threats e.g. for an estimated 44 million men and women older than age 50 in the United States. Those 44 million comprise approximately 55% of all persons aged 50 and older; the number of those affected is estimated to increase to 52 million by 2010 and to 61 million by 2020. Women. are at a much greater risk than men for having low bone mass and developing osteoporosis. Approximately 80% of those with osteoporosis are women. Worldwide, the number of women with osteoporosis will increase from 30 million in 2002 to 35 million in 2010 and to nearly 41 million in 2020. Although osteoporosis is often considered a “woman's disease,” and women are more prone to develop it, men also are susceptible to the disease. It is estimated that there are more than 2 million men in the United States who currently have osteoporosis. The number of cases is expected to increase to nearly 3 million in 2010.

Consequences of Osteoporosis

The most devastating consequence of osteoporosis is fractures. More than 1.5 million fractures occur annually—700,000 vertebral, 300,000 hip, and 200,000 wrist—and nearly 50% of vertebral fractures go undiagnosed. One of every 3 women and 1 of every 8 men will have an osteoporosis-related fracture during their lifetime.

Postmenopausal women have a greater risk for fracture than any other sample population: 15% fracture a hip and 20% sustain a vertebral fracture. In women, the annual number of osteoporosis-related fractures exceeds the combined incidence of heart attack, stroke, and breast cancer. Although a single fracture may be devastating in itself, it is important to note that patients experiencing a first fracture are at a nearly 5-fold increased risk for subsequent fractures; these patients are often considered to have entered a “cascade of fractures.”

The morbidity of a fracture is apparent at the time of the incident; however, fractures also are associated with increased mortality. Hip fractures are regarded as the most severe complication of osteoporosis. After a hip fracture, 20% of patients die and more than 50% of the survivors require long-term nursing home care. It is estimated that one fifth to one third of all hip fractures occur in men. Men, although they are less likely to have osteoporosis, have higher mortality rates due to hip and spinal fractures than women with osteoporosis. Seventeen percent of all men have had a hip fracture by age 90 compared with 32% of women.

Diagnosis

There are 5 major risk factors for the development of osteoporosis. As a person ages, bone resorption increases (e.g., in early postmenopausal women) or remains stable, whereas bone formation rates decrease. This can lead to low bone mass and eventually to osteoporosis. Women are more prone to osteoporosis because of several physical and genetic factors. Women have a lower peak bone mass, a lower muscle mass, and a smaller periosteal diameter of their bones. Women also lose bone during their reproductive years, especially during prolonged lactation (although this is reversed after lactation ceases), and life expectancy is greater in women. Collectively, these elements increase skeletal fragility in women. Osteoporosis is more prevalent in white and Asian populations because of the lower peak bone mass in these populations. The reason for this prevalence is not completely understood. Heredity plays a major role in the determination of osteoporosis-related problems. Approximately 50% to 60% of peak bone mass is genetically determined. Differences in specific genes for collagen, hormone receptors, and local factors contribute to the risk for osteoporosis. The actual body weight of an individual can affect risk. The conversion of androgens to estrogens, which takes place in fat tissue, occurs less in a person with a thin body habitus (physical frailty). Conversely, an obese person has increased muscle mass and more subcutaneous fat, which affords the skeleton greater protection. Decreased muscle mass and oestrogen levels are considered potential risk factors. Minor risk factors include systemic hormone levels, local factors, co-morbid conditions, and social history.

Bone mineral densitometry is the only definitive method to diagnose osteoporosis, determine bone density, assess fracture risk, and monitor response to therapy. A bone mineral density BMD test is a painless, non-invasive, safe, and readily available procedure. Traditional tests evaluate bone density in the spine, hip, and wrist; however, BMD tests also can be performed on the finger, heel, and shinbone. Results of a BMD test are compared with “young-normal” and “age-matched” values. The young-normal value (T-score) represents the average optimal density of a 20-to-30-year-old adult. The age-matched score (Z-score) represents the average value of someone of the same sex, age, and body size as the test recipient. The American Association of Clinical Endocrinologists recommends that all women 65 and older, those with a history of fracture, and younger postmenopausal women who have clinical risk factors for fracture should be tested for bone mineral density.

A number of BMD tests currently are available. Generally, BMD tests are either a full-body scan (i.e., measures bone density in the hip and spine) or a peripheral scan (i.e., measures bone density in the finger, wrist, or heel). Dual-energy x-ray absorptiometry (DXA) is considered the gold standard of BMD tests. The DXA, although simple to perform and widely available, cannot quantify trabecular and cortical bone mineral density separately. However, peripheral quantitative computed tomography (pQCT) is a more powerful technique that can quantify bone mineral density in trabecular and cortical bone. This technique measures bone mineral density in the extremities in 3 dimensions and eliminates many of the artefacts associated with DXA. However, it is not widely available.

Drug Therapy

Preventive pharmacologic management should be considered for all patients with risk factors or healthy subjects at risk of osteoporosis at a later stage. Calcium and vitamin D supplements have been shown to increase BMD and often are used in conjunction with other therapeutic agents. Medications used to prevent and treat osteoporosis belong to 2 categories: antiresorptive agents and anabolic agents. There are several antiresorptive agents on the market, including oestrogen replacement therapy (ERT), bisphosphonates, selective oestrogen receptor modulators (SERMs), and calcitonin. ERT has been considered the standard initial treatment for postmenopausal women, those with early menopause, and women with surgically induced menopause. The addition of a progestin to ERT prevents endometrial hyperplasia and decreases the risk for uterine malignancy, as well as prevents bone loss. Recently, safety concerns regarding oestrogen therapy have been raised after interim results from the Women's Health Initiative associated increased risk for breast cancer, myocardial infarction, and stroke with oestrogen therapy. Since these results, the usage of estrogens for osteoporosis has decreased, which emphasizes the need for other osteoporosis treatment options.

Bisphosphonates are considered first-line therapy for patients who have already experienced a fracture or have a high degree of bone loss. They also are an option for women in whom estrogens are contraindicated. Bisphosphonates are potent antiresorptive drugs that increase bone mass and decrease the risk for fractures. Both alendronate and risedronate have been shown to significantly decrease the incidence of vertebral and non-vertebral fractures, including fractures of the hip. Alendronate and risedronate are indicated for the prevention and treatment of postmenopausal osteoporosis and glucocorticoid-induced osteoporosis in men and women. The physicians are encouraged to ensure adequate supplementation of calcium and vitamin D for the patients. Alendronate is also indicated for the treatment of osteoporosis in men. In general, bisphosphonates are poorly absorbed (<1%), necessitating administration in the absence of food or other medications. Although the introduction of once-weekly formulations has reduced the inconvenience of the strictly regimented administration, bisphosphonates are associated with gastrointestinal adverse effects. A number of new bisphosphonates are in development for the prevention and treatment of osteoporosis, including ibandronate , zoledronic acid, minodronate, and neridronate. These agents may provide bisphosphonate options that are better tolerated and more conveniently administered.

SERMs are antiestrogenic in classic organs (e.g. breast), but have antiresorptive effects on bone, as well. The only approved agent in this class for the prevention and treatment of postmenopausal osteoporosis is raloxifene. It is widely popular among gynecologists; however, its efficacy data are somewhat less compelling than those of the bisphosphonates. BMD increases are less than those seen with the bisphosphonates, and studies have not demonstrated significant reductions in nonvertebral fractures. Potential advantages of these agents include a reduced risk for breast cancer and positive cardiovascular parameters. Raloxifene is generally well tolerated; however, there are risks (hot flashes, thrombosis) that are not associated with bisphosphonate therapy. There are a number of new SERMS in development, including bazodoxifenedoxifene, and lasofoxifene.

Calcitonin is used as an antiresorptive and is currently available as a nasal spray or subcutaneous injection. Although calcitonin is indicated for the management of postmenopausal osteoporosis to prevent progressive loss of bone mass and the treatment of glucocorticoid-induced osteoporosis, evidence of its efficacy in preventing fractures is inconclusive. In general, calcitonin is considered a less effective agent by most clinicians.

Anabolic agents stimulate bone formation. Teriparatide (PTH 1-34), the only anabolic agent currently available, is used in the treatment of osteoporosis in postmenopausal women who are at high risk for fractures (including those with a history of osteoporotic fracture, those with multiple risk factors for fractures, and those intolerant of or failing to respond to prior therapy for osteoporosis). It also is indicated for osteoporosis in men with hypogonadism in the US but not yet in Europe. Teriparatide is the N-terminal fragment of recombinant PTH. In clinical trials, teriparatide has been shown to significantly increase bone mineral density and to reduce the risk for vertebral and some nonvertebral fractures. All patients including the ones in control and for placebo groups were provided calcium and vitamin D treatment. Aside from the inconvenience of its subcutaneous administration, teriparatide is generally well tolerated, with the most common adverse effects being nausea, headache, hypercalcemia and hypotension. The drug is contraindicated in patients with open epiphyses (i.e. children, adolescents), Pagets disease of the bone, prior radiation therapy involving the skeleton, bone metastases or skeletal malignancies, metabolic bone diseases other osteoporosis, or pre-existing hypercalcaemia.

Full length PTH 1-84 is identical to the endogenous 84-amino acid human PTH that is synthesized and secreted by the parathyroid glands, and has likewise been developed for the treatment of osteoporosis. Studies support the development of full-length PTH primarily assessed by the effects of 1-84 on bone formation, mass architecture and strength.

Strontium ranelate, composed of an organic moiety (ranelic acid) and 2 atoms of stable non-radioactive strontium and formulated as a powder to be taken orally, is currently in clinical development for the treatment of osteoporosis. Results from Phase III clinical trials, in which also calcium and vitamin D treatment was given to all patients, have suggested that it is effective in increasing BMD and reducing the risk for both vertebral and non-vertebral osteoporosis, while being generally well tolerated.

Pharmaceutical Compositions

As mentioned herein before, any suitable principle may be applied such as those mentioned above, especially using a combination of two or more types of pellets/granules having different release pattern. In the following is given a specific suitable technology developed by the present inventors to deliver active substances to the small intestine or the colon. The individual pharmaceutically acceptable excipients mentioned below may also be applied in other types of compositions; a person skilled in the art will know how to select suitable excipients depending on the particular composition. Another type of composition of particular interest is an enteric coated composition, e.g. an enteric coated tablet or capsule.

The present invention provides a pharmaceutical composition that provides a predetermined lag time before the active substance is released. The lag time obtained is based on a combination of two principles, namely a combination of a pH dependent release and/or a pH independent, but time controlled release.

In contrast to many of the known colon delivery systems, the pharmaceutical composition according to the present invention is contemplated to be suitable for large-scale production.

Thus, the present invention provides a pH and/or time-controlled pharmaceutical composition for oral use comprising one or more of a first type of unit, the first type of unit comprising PTH and having a layered structure of at least

i) an inner core

ii) a time-controlled layer surrounding the Inner core,

iii) a film coating applied on the time-controlled layer, wherein the film coating is substantially water insoluble but permeable to an aqueous medium, and

iv) an outer layer of an enteric coating.

The release of PTH from the unit—when tested in vitro as an average of at least six determinations—is not more than about 10% w/w at a first pH value below about 4.0, and at a second pH value of from about 5.0 to about 8.0 the active substance is released in such a manner that—after a lag time of from about 0.5 to about 8 hours in which first time period not more than about 10% w/w of the active substance is released—at least about 50% w/w of the active substance contained in the unit is released within a second time period of not more than about 2 hours.

The pharmaceutical composition may be in the form of a multiple unit composition comprising a multiplicity of individual units or it may be in the form of a single unit composition. In the case of a multiple unit composition, the pharmaceutical composition may contain more than one type of unit. Thus, in order to obtain a composition with a specific release pattern of the active substance, the composition may contain a mixture of two or more types of units each having a specific release pattern of the active substance.

The active substance, PTH, is contained in the unit in one or more of the layers i)-iii) and/or in a further layer v) surrounding the inner core. In a specific embodiment of the invention the active substance is contained in the further layer v) and normally, the further layer v) is situated between layer i) and ii).

As mentioned above, a pharmaceutical composition according to the invention is especially suitable when the active substance is subject to colon absorption and/or exerts its effect in the colon.

The present inventors have found that in order to obtain a pharmaceutical composition that enables a predetermined delay in the release of the active substance and at the same time enables a relatively fast release of the active substance after the predetermined delay, it is suitable to take advantage of two different principles for delaying the release of the active substance, namely one principle for the delay in those parts of the gastrointestinal tract wherein the pH is in the acidic region and another principle for the delay in those parts of the gastrointestinal tract, wherein the pH is in the neutral and alkaline region.

The principle employed in those parts of the gastrointestinal tract wherein the pH is in the acidic region is based on the enteric coating principle, i.e. the possibility of providing a coating that is substantially insoluble in an acidic environment, but which is soluble in a neutral and alkaline environment. This is achieved by use of so-called enteric polymers, which are insoluble in acidic media, but soluble in neutral and alkaline media. Accordingly, the release is dependent on a shift of pH from the acidic region to the neutral/alkaline region.

The individual and inter-individual variations with respect to gastric emptying are therefore of minor importance when a pharmaceutical composition according to the invention is applied. Furthermore, in those cases, where the pharmaceutical composition is in the form of a multiple unit composition the gastric transit time of the multiple units is normally relatively independent of whether the patient is in fasted or fed state. This is in contrary to what is generally seen when a single unit composition is administered.

In a specific embodiment of the invention, the enteric polymer employed is a polymer that has a pH cut off that enables the start of the dissolution of the enteric coating at the time when the delivery system enters the small intestine. In the present context the term “pH cut off” is defined as the lowest pH value by which the enteric polymer is soluble at a temperature of 37° C. In contrast to the transit time in the stomach, the transit time in the small intestines is relatively constant (3-5 hours). The present inventors have therefore found that it is an advantage to design a delivery system that independently of the transit time in the stomach has properties that governs when the release of the active substance takes place after entering into the small intestine.

The principle employed in those parts of the gastrointestinal tract, wherein the pH is in the neutral/alkaline region, is based on a time controlled release. Whereas the pH in the stomach normally is about 1.5-2.0 for fasted conditions and about 3.0-5.0 for fed conditions, the pH of the small intestine is about 5.0-6.5 in the jejunum, about 6.0-7.5 in the ileum and about 6-8 in the colon. The variation of pH in the intestine is difficult to use from a pharmaceutical formulation point of view, but the relatively constant transit time in the small intestine is a much more favourable approach. Accordingly, a pharmaceutical composition according to the present invention is designed so that after entry into the small intestines the enteric coat is relatively fast dissolved and a time controlled process is started by which the time controlled layer contained in the unit is controllable subject to a process that results in the breakage of the film coating layer. In those cases where the time controlled layer is a swellable layer, the layer starts to swell. At a certain point in time the swellable layer has swelled to such an extent that the film coating layer, that coats the swellable layer, breaks, disrupts or is otherwise destroyed. Then the active substance contained in the unit becomes exposed to the gastrointestinal tract and is ready to be absorbed or to exert its effect either immediately or later.

pH Dependent Release—Enteric Coating

As mentioned above, the unit(s) contained in a pharmaceutical composition of the present invention is (are) coated with an enteric coating. Normally, this coating is the outermost layer of the unit(s).

As mentioned above, the term “pH cut off” is intended to indicate the lowest pH value at which the enteric polymer is soluble at a temperature of 37° C. The pH cut off of the enteric polymer is important in order to ensure that the enteric coating is dissolved as quickly as possible after entering of the pharmaceutical composition into the small intestine.

Accordingly, the enteric coating for use in the present invention comprises an enteric polymer that has a pH cut off of at the most about 8.0 such as, e.g. in a range of from about 4 to about 7.5, in a range of from about 4.5 to about 7.0, from about 4.9 to about 6.9, from about 5.0 to about 6.5, from about 5.0 to about 6.3, from about 5.0 to about 6.0, from about 5.0 to about 5.9, from about 5.0 to about 5.7, from about 5.0 to about 5.6 or from about 5.0 to about 5.5.

The enteric coating used according to the invention comprises an enteric polymer. Suitable enteric polymers are selected from the group consisting of e.g.:

Amylose acetate phthalate, cellulose acetate phthalate CAP (pH cut off about 6.2), cellulose acetate succinate, cellulose acetate trimellitate CAT (pH cut off about pH 5.0), carboxymethyl ethylcellulose, formalin treated gelatine, hydroxypropyl methylcellulose acetate succinate HPMCAS (pH cut off about 5.0-5.5), hydroxypropyl methylcellulose acetate phthalate, hydroxypropyl methylcellulose phthalate HPMC-P (pH cut off about 5.0 and about 5.5), methacrylic acid copolymer (Eudragit L) (pH cut off about 5.5 and about 6), methacrylic acid copolymer (Eudragit S) (pH cut off about 7), methacrylic acid copolymer (Eudragit FS) (pH cut off about 7.5), polyvinyl acetate phthalate PVAP (sureteric), shellac, starch acetate phthalate, styrene-Maleic acid copolymer, zein, and mixtures thereof.

Normally, the concentration of the enteric polymer used is in a range corresponding to about 2 to about 60% w/w based on the total weight of the unit. The enteric coating may also contain additives like those mentioned herein later. Thus, e.g. plasticizers etc. may be suitable as additives.

The Cores

The inner core of a pharmaceutical composition according to the invention may be an inert core or a core containing the active substance. It may also be in the form of a pellet, granules, granulates or a tablet. In the latter case, the pharmaceutical composition is presented in the form of a single unit composition.

Examples of a core suitable for use according to the invention are, e.g., calcium alginate beads, cellulose spheres, charged resin spheres, glass beads, polystyrene spheres, sand silica beads or units, sodium hydroxide beads, sucrose spheres, collagen-based beads and crystals of an active substance.

In general, a person skilled in the art can find guidance and advice of how to formulate and perform individual process step in Remington's Pharmaceutical Handbook to which reference is made.

Time Controlled Release

The time controlled release that is intended to start when the delivery system enters the small intestine is based on the idea that a film coating layer to a certain degree essentially prevents any active substance to be release from the composition until the film coating layer is impaired. The properties of the film coating layer is that it is essentially insoluble in water or aqueous media, but it permits penetration of water or aqueous media into the composition (but not as long the enteric coating is present; the enteric coating is essentially not permeable to water). The water or aqueous media that diffuse into the system may dissolve some of the active substance that is contained within or inside the film coating layer and an outward oriented diffusion process of the active substance may be operating. However, if this is the case, the end result must be that the transport of active substance out of the system via the film coating is very slow and at the most about 10% w/w of the active substance is released by such a process.

The time controlled layer may comprise a substance that is swellable, osmotic and/or effervescent. In a specific embodiment, the time controlled layer is a swellable layer.

The purpose of the time controlled layer is that upon entering of water into the layer, a process starts that results in disruption or breakage of the film coating membrane. The mechanism by which this process operates may be a swelling process, an osmotic pressure driven process and/or a process based on effervescence. A combination of these mechanisms may also be operating.

The intrusion of water into the time controlled layer may also start the dissolution process of an active substance contained in the layer or in another layer inside the time. This may be an advantage in those cases where the active substance is not readily soluble in water or where it has a relatively slow dissolution rate.

The intention of the combination of a time controlled layer such as, e.g., a swellable layer and a film coating layer is that a swelling process of the swellable layer starts when the water or aqueous media starts to diffuse into the system through the film coating. The swellable layer is able to adsorb/absorb a specific amount of water and to expand in size. When a certain size of the swellable layer is obtained, the film coating will no longer be flexible enough to withstand any disruption and it will break, explode or be destroyed.

In this manner a predetermined lag time may be obtained by controlling the time it takes for the swellable layer to swell to such an extent that the film coating layer is disrupted or destructed. In the case of an osmotically active layer (in those cases where the time controlled layer predominantly contains an osmotically active substance) and an effervescent active layer, the end result is the same as mentioned above, namely disruption or breakage of the film coating layer.

The lag time may be adjusted by careful selection of i) the specific composition of the time controlled layer, ii) the thickness or amount of the time controlled layer, iii) the specific composition of the film coating layer and/or iv) the thickness of the film coating layer. Suitable additives may be added to the time controlled layer and/or the film coating layer in order to adjust the lag time.

In a delivery system according to the invention, the film coating normally comprises a water insoluble polymer selected from the group consisting of e.g.:

Ammonio methacrylate copolymer (Eudragit RL, Eudragit RS), cellulose acetate, cellulose acetate butyrate, cellulose acetate propionate, cellulose butyrate, cellulose propionate, cellulose valerate, crospovidone, ethyl cellulose, hydroxypropylcellulose, hydroxyethylcellulose, polyacrylate dispersion (Eudragit NE), polydiethylaminomethylstyrene, polymethylstyrene, polyvinyl acetate, polyvinyl formal, polyvinyl butyryl, wax, and mixtures thereof.

In a specific embodiment, the water insoluble polymer creates a relatively non-flexible film coating. This may be obtained by application of a polymer that has a relatively short chain length and/or by avoiding any or excessive amount of plasticizer.

In a further embodiment, the film coating layer iii) comprises ethyl cellulose and/or hydroxypropylcellulose. As mentioned above, short chain length polymers are suitable for use such as, e.g., ethyl cellulose that has a viscosity of at the most about 20 cps.

In those cases, where it is desired to ensure a fast destruction of the film coating layer when the swellable layer has exceeded a certain size, it may be suitable to employ a film coating layer iii) that further comprises an additive that promotes disruption or destruction of the film coating layer upon exposure to an aqueous medium.

Suitable additives may be selected from the group consisting of e.g.:

Acetylated monoglyceride, acetyltributyl, acetyltributyl citrate, acetyltriethyl citrate, benzyl benzoate, calcium stearate, castor oil, cetanol, chlorebutanol, colloidal silica dioxide, dibutyl phthalate, dibutyl sebacate, diethyl oxalate, diethyl malate, diethyl maleate, diethyl malonate, diethyl fumarate, diethyl phthalate, diethyl sebacate, diethyl succinate, dimethylphthalate, dioctyl phthalate, glycerin, glyceroltributyrate, glyceroltriacetate, glyceryl behanate, glyceryl monostearate, hydrogenated vegetable oil, lecithin, leucine, magnesium silicate, magnesium stearate, paraffin, polyethylene glycol, propylene glycol, polysorbate, silicone, stearic acid, talc, titanium dioxide, triacetin, tributyl citrate, triethyl citrate, zinc stearate, wax, saturated fatty acids and mixtures thereof.

In a specific embodiment, a suitable additive is a polyethylene glycol, magnesium stearate and/or paraffin. The polyethylene glycol may be, e.g., PEG 200, 300, 400, 540, 600, 900, 1000, 1450, (1500)1540, 2000, 3000, 3350, 4000, 4600, 6000, 8000, 20000, or 35000. PEGs having a molecular weight of from about 200 to about 600 are liquids, whereas PEGs having a molecular weight of 1000 and above are solids.

The time controlled layer ii) of a pharmaceutical composition of the invention normally comprises a swelling agent, an osmotically active agent and/or an effervescent agent.

The time controlled layer can also comprise one or more pharmaceutically acceptable excipients.

A swelling agent for use according to the invention may be selected from the group consisting of e.g.:

Alginic acid, alginates, carboxymethylcellulose calcium, carboxymethylcellulose sodium (Ac-Di-Sol), crospovidone, hydroxypropylcellulose, hydroxypropylmethylcellulose (HPMC), low substituted hydroxypropylcellulose (L-HPC), microcrystalline cellulose, polacrilin potassium, polyacrylic acid, polycarbofil, polyethylene glycol, polyvinylacetate, polyvinylpyrrolidone, polyvinylpyrrolidone, plasdone, sodium croscarmellose, sodium starch glycolate (Explotab), starches, and mixtures thereof.

In those cases when the time-controlled layer ii) comprises an effervescent agent, such an agent is typically selected from alkali metal carbonates, alkali metal hydrogen carbonates, alkaline earth metal carbonates, alkaline earth metal hydrogen carbonates, citric acid, tartaric acid, fumaric acid, etc., and mixtures thereof.

When the time-controlled layer ii) comprises an osmotic agent it is e.g., sodium chloride and/or sorbitol.

Normally, the weight fraction of the time controlled layer is from about 25% to about 90% w/w based on the weight of the total unit.

Active Substances

The term “active substance” encompasses the active substance in any suitable form.

Thus the active substance may be present in the form of a pharmaceutically acceptable salt, complex or prodrug thereof, or, whenever relevant, it may be present in racemic or any of its enantiomeric forms. Furthermore, it may be present in solid, semi-solid or dissolved form such as, e.g. in the form of particulate material e.g. in the form of crystals or it may be present in any amorphous or polymorphous form. Furthermore it may be presented as micronised powder or in the form of a solid dispersion.

Examples of active substances for use in a pharmaceutical composition according to the invention are generally any active substance that is therapeutically, prophylactically and/or diagnostically active. As mentioned hereinbefore, a PTH is a mandatory active substance in a composition according to the invention and besides PTH, other active substances normally used in the prevention or treatment of bone related disorders could be employed.

More specifically, active substances within the below-mentioned classes are especially suitable for use in a pharmaceutical composition according to the present invention. The specific examples of active substances mentioned below are only for illustrative purposes and are not construed to limit the invention in any way. They illustrate other active substances that are suitable for use in bone related disorders. It is possible to include other active substances in a composition of the invention and such a substance can be found outside the below-given classification.

Statins

The statins (e.g. atorvastatin, cerivastatin (rivastatin), dalvastatin, lovastatin, fluvastatin, glenvastatin, pitavastatin (itavastatin, nisvastatin), pravastatin (eptastatin, epastatin), rosuvastatin, simvastatin (epistatin, synvinolin, velostatin) and tenivastatin competitively inhibit 3-hydroxy-3-methylglutaryl coenzyme A (HMG CoA) reductase, an enzyme involved in cholesterol synthesis, especially in the liver. They are more effective than other classes of drugs in lowering LDL-cholesterol but less effective than the fibrates in reducing triglycerides and raising HDL-cholesterol. Statins produce important reductions in coronary events and in all cardiovascular events. Statins have a role in primary prevention of coronary events in patients at increased risk.

Bone Effects

Experimental evidence based on retrospective studies suggests that the cholesterol-lowering drugs statins may increase bone formation shown by a significant increase of bone-mineral density associated with taking statins in postmenopausal women (Edwards C J et al. Lancet 2000; 355: 2218-2219; Lupattelli G et al. Metabolism. 2004 Jun; 53(&): 744-8).

Other Effects

Statins appear to have favourable impact on psychological conditions for elderly patients with coronary disease who take statins over the long term and show improvements in psychological disorders.

Other examples are:

Antiresorptive agents to be administered on a daily, weekly, quarterly, half-yearly or yearly basis are for example but not limited to:

Bisphosphonates e.g. Ibandronate, Pamidronate, Alendronate, Zoledronic acid Risedronate, Tiludronate, Etidronate, Minodronate.

Selective estrogen receptor modulators (SERMs) e.g. Raloxifene, Lasofoxifene, Bazodoxifene, Arzoxifene, Ospemifene

Hormone Replacement Therapy e.g. Tibolone

Calcium regulating agents e.g. Calcitonine

Strontium ranelate

Cathepsine K inhibitors

Glucocorticoides e.g. Prednisolon, Budesonide

Anti-androgen agents e.g. Flutamide

Other agents of interest are e.g. Folic acid, Pravastatin, Ranithidine, Danazole, Vitamin B12, Calcium, Vitamin K

The amount of the specific active substance in a pharmaceutical composition according to the invention depends on the condition to be treated and on the age and condition of the patient. Moreover it depends on the frequency of the dosing, i.e. on the system is intended for use 1, 2, 3, 4, 5 or more times daily, weekly, monthly, quarterly, half yearly or yearly. A person skilled in the art will know how to decide the correct dosage in a pharmaceutical composition of the invention.

In the case of a composition containing PTH, a person skilled in the art will know which dose to include in the composition based on clinical relevant data.

The same applies in the case of a composition containing PTH in combination with a calcium compound and/or a vitamin D.

Pharmaceutically Acceptable Excipients and Other Additives

A pharmaceutical composition according to the invention may further comprise one or more pharmaceutically acceptable excipients. The use of pharmaceutically acceptable excipients is well-known in the art of pharmaceutical formulation and may be employed e.g. to facilitate the manufacturing process and filling of the delivery system into a suitable dosage form (e.g. capsules, sachets etc.).

Suitable pharmaceutically acceptable excipients are selected from the group consisting of fillers, diluents, binders and sweeteners.

Specific examples include:

Agar, alginate e.g. sodium alginate, calcium bicarbonate, calcium carbonate, calcium hydrogen phosphate, calcium phosphate, calcium sulphate, carboxyalkylcellulose, cellulose, charged sodium polystyrene sulphonate resin, dextran, dextrates, dextrin, dibasic calcium phosphate (Emcompress), ethyl cellulose, gelatine, glucose, glyceryl palmitostearate, gummi arabicum, hydroxyethyl cellulose, hydroxypropyl cellulose, hydroxypropylmethylcellulose, magnesium carbonate, magnesium chloride, magnesium oxide, maltodextrin, methylcellulose, microcrystalline cellulose, modified starches, polyethylene glycol, polyethylene oxide, polysaccharides e.g. dextran, polyvinylpyrrolidone (PVP), polyvinylpyrrolidone/vinyl acetate copolymer, soy polysaccharide, sodium carbonate, sodium chloride, sodium phosphate, starch, dextrose, fructose, glycerin, glucose, isomalt, lactitol, lactose, maltitol, maltose, mannitol, aorbitol, sucrose, tagatose, trehalose, xylitol, alitame, aspartame, acesulfam potassium, cyclamic acid, cyclamate salt (e.g. calcium cyclamate, sodium cyclamate), neohesperidine dihydrochalcone, thaumatin, saccharin, saccharin salt (e.g. ammonium saccharin, calcium saccharin, potassium saccharin, sodium saccharin), sucralose and mixtures thereof.

One or more excipients may also be added in order to improve the stability, the taste, the storage time etc. of the composition (or the active substance(s) contained in the composition) or to improve the bioavailability of the active substance(s) including the dissolution rate, the absorption rate and the extent of absorption. To this end incorporation of an enhancer is suitable. In the following is given a number of examples on enhancers suitable for use in a composition of the present invention. Although the discussion is focused on peptides, the enhancers may suitably be used for any active substance for which an improvement in absorption is desired. Thus, the discussion below is not intended to limit the invention in any way. In those cases, where an enhancer is present in a composition of the invention, it can be incorporated in any of the layers contained in the composition. Normally, it is incorporated in the layer containing the active substance for which absorption should be enhanced or in a layer in close proximity to this.

Absorption Enhancers and Stabilising Agents Including PTH Stabilising Agents

The absorption of peptides and proteins in the gastrointestinal (GI) tract is low, because the absorption depends on various important factors: the size, instability in the GI-tract etc. The peptides and proteins can be chemical deactivated by different proteases. The absorption may be improved by use of enzyme inhibitors, which may result in the deactivations of the enzymes (proteases). However, enzyme inhibitors might be absorbed and trigger several side effects including systemic toxicity. In general, low molecular weight absorption enhancers disrupt the mucosal layer of the gut tissue. There is therefore a risk that enhancement in absorption of peptides and proteins can be accompanied by toxic effects of such enhancers. Another way to improve the oral absorption is to increase the stability of peptides and proteins in the GI-tract by chemical modification.

It is therefore essential to ensure that, by opening tight junctions, the enzyme inhibitors and absorption enhancers are not absorbed together with the peptides or proteins. Also it is important to ensure that other proteins originating from the ingested meal do not get absorbed or causes toxic effects when exposed systemically.

Carrier systems are necessary to increase the residence time of the delivery system for a specific period of time or to delivery of the peptides or proteins at a desirable absorption site in the GI-tract, during which the peptides or proteins can be released and absorbed. These carrier systems should not essentially influence the physicochemical properties of the peptides or proteins.

Below are lists different types of substances that are suitable for use in a composition according to the invention in order to improve the absorption of one or more active substances in particular of peptides and proteins either by inhibiting enzymes or enhancing the absorption of peptides and proteins.

Enzyme inhibitors, for example

Protease inhibitors (e.g. Aprotinin, Amastatin, Carboxyl Esterase, Carboxymethylcellulose-Bowman-Birk, Carboxymethylcellulose-Elastatinal, Chicken Ovomucoid, Chymostatin, Duck Ovomucoid, Lactate dehydrogenase, Leupeptin, Bestatin, α2-Macroglobulin, Soybean Trypsin)

Effective concentrations of these compounds might vary depending on the compounds, examples are Aproitinin; high dose concentration 0.5 mg/ml to 2 mg/ml, low concentration 0.125 mg/ml and Amastatin; High concentration is 0.03 mg/ml and low concentrations are 0.005 mg/ml

Chelating agents (e.g. Ethylenediaminetetraacetic Acid (EDTA), Chitosan-EDTA, Chitosan-EDTA-Antipain, Chitosan-EDTA-Chymostatin, Chitosan-EDTA-Elastatinal, Chitosan-EDTA-Bowman-Birk inhibitor)

Various polymers (e.g. Carbomer, Chitosan, Chitosan-Antipain, Chitosan-Chymostatin, Chitosan-Elastatinal, Chitosan-DTPA (DTPA=DiethyleneTriaminePentaacetic Acid), Polycarbophil)

Of the above-mentioned enzyme inhibitors, Chitosan-EDTA, Chitosan-EDTA-Antipain, Chitosan-EDTA-Chymostatin, Chitosan-EDTA-Elastatinal, Chitosan-EDTA-Bowman-Birk inhibitor, Chitosan-Antipain, Chitosan-Chymostatin, Chitosan-Elastatinal, Chitosan-DTPA are especially suitable for use because enzymes inhibitors with low molecular weight such as Aprotinin or EDTA might be absorbed easily and may cause side effects as systemic toxicity. It is possible to avoid their systemic absorption and to exclude side effects (e.g. by covalent attach the enzyme inhibitors to unabsorbable hydrophilic matrices of high molecular weight or polymers with mucoadhesive properties (e.g. Chitosan)). Additionally, this approach may increase the luminal concentration and result in more effective inactivation of the enzymes.

Absorption Enhancers

Ideally, appropriate absorption enhancers for use in a composition of the invention should have the following properties. A) Compatible with peptides and proteins with respects to possible chemical interaction, which might change the physicochemical structure and pharmacological activity of the peptides and proteins. B) Rapid response to open the tight junctions. C) Afford therapeutic levels of peptides or proteins in the systemic circulation. D) Rapid reversible effect to close the tight junctions in order to diminish probable side effects by avoiding the uptake of unwanted toxic substances in the intestine.

Fatty acids and surfactants increase the epithelial membrane permeability by interacting with the phospholipids bilayer of the intestinal membranes and may cause toxic side effects in the cells.

Fatty acids, fatty alcohols and fatty esters, for example:

Ethyl Oleate, Sodium Oleate, Lauric Acid, Methyl Laurate, Oleic Acid, Sodium Caprate

Surfactants, for example:

Dioctyl Calcium Sulfosuccinate, Dioctyl Potassium Sulfosuccinate, Dodecyltrimethylammonium Bromide, Glyceryl Monooleate, Hexadecyltrimethylammonium Bromide, Trimethyltetradecylammonium Bromide, Polyoxyehtylene Ethers (Polyoxyehtylene-9-lauryl Ether), Polysorbates, Sodium Dodecyl Sulphate, Sodium Dioctyl Sulfosuccinate, Sodium Laurate, Sodium Lauryl Sulfate, Sodium 5-methoxysalicylate, Sodium Salicylate, Sorbitan Esters

Selective absorption enhancers of a high molecular weight, such as anionic polyacrylates and cationic chitosans, may manage to selectively open the tight junctions. In addition to the ability of mucoadhesive substances to bind unspecifically to mucus, they may also increase paracellular permeability and inhibit the action of proteolytic enzymes. The increased paracellular permeability may allow not only the active substance but also toxic substances to be absorbed into the systemic circulation. Chitosan and its derivates (e.g. N-Trimethyl Chitosan Chloride) are known as potential absorption enhancers for peptides and proteins. They manage to selectively open the tight junctions to allow the passive absorption of peptides and proteins via the paracellular pathway. They display mucoadhesive properties and enhance the interaction of the delivery systems with the intestinal mucosa to prolong the duration of absorption.

Mucoadhesive polymers, for example:

Alginate, Cellulose derivates (e.g. Carboxymethylcellulose, Methylcellulose, Hydroyethyl Cellulose, Hydroxypropyl Cellulose, Hydroxypropyl Methylcellulose, Sodium Carboxymethylcellulose), Carbomer, Carbopol (Polyacrylic Acid), Carbopol-PEG, Chitin, Chitosan (α(1-4)2-amino 2 deoxy β-glucan), Trimethyl Chitosan, N-Trimethyl Chitosan Chloride, Poly(acrylamide), Polyacrylates (e.g. Poly(alkyl cyanoacrylate), Poly(butyl cyanoacrylate), Polyethylene Glycol, Polyethylene Oxide, Poly(ethyl cyanoacrylate), Poly(2-hydroxyethyl methacrylate), Poly(isobutyl cyanoacrylate) Poly(isohexyl cyanoacrylate), Poly(methyl methacrylate)), Poly(D,L-lactic acid), Poly-DL-Lactide-poly(ethylene glycol), Poly(lactic acid-co-glycolic acid), Polyanhydrides (e.g. Poly(fumaric anhydride), poly(fumaric-co-sebacic anhydride)), Poly(vinyl alcohol), Polycarbophil, Polycarbophil-Cysteine, Poly(methylmethacrylate), Povidine-(polyvinylpyrrolidone), Starch (e.g. Amylose, Amylopectin), Sodium Hyaluronate, Hyaluronic acid, Thiolated polymers (Thiomers)). Of particular interest are Chitosans

Bile salts enhance the transmembrane transport of endogenous and exogenous lipophilic compounds as well as the paracellular transport of polar hydrophilic molecules across the intestinal epithelium.

Bile salts, for example:

Sodium Deoxycholate, Deoxycholic Acid, Sodium Cholate, Cholic Acid, Sodium Glycocholate, Sodium Glycodeoxycholate, Sodium Taurocholate, Sodium Taurodeoxycholate

Cytoadhesives bind specifically—via a receptor-ligand-like interaction—to the surface of the epithelial cells. They may transmit signals, which induce substrate-specific vesicular transport processes. From a toxicological point of view these specific transport processes may be preferred to the general increase of permeability offered by some mucoadhesives. Lectins are protein or glycoproteins of nonimmunological origin, which specifically recognise sugar molecules and therefore are capable of binding to glycosylated membrane components.

Cytoadhesives, for example:

Lectins (e.g. Lycopersicon Esculentum Agglutinin, Wheat Germ Agglutinin, Urtica Dioica Agglutinin).

A new family of low molecular weight carriers, derived from N-acylated amino acids, has been developed and are also useful in the present context. They are thought to increase selectively the mucosal uptake by inducing conformational changes in the peptide molecules. While forming non-covalent bonds with the carrier, the molecules undergo partial unfolding and may both relax their shape and expose inner lipophilic residues thus facilitating their transmembrane passage. Unlike traditional surfactants and detergents, this class of absorption enhancer has certain specificity for peptides and proteins and polyaminoglycans and is practically devoid of toxic activity toward the intestinal epithelial cells.

N-acylated Amino Acids (especially N-[8-(2-hydroxy-4-methoxy)bensoyl]amino Caprylic Acid (4-MOAC), 4-[4-(2-hydroxybenzoyl)amino]butyric Acid, Sodium N-[8-(2-hydroxybenzoyl)amino]-caprylate)

Various other suitable absorption enhancers are listed below.

Phospholipids, for example:

Hexadecylphosphocholine, Dimyristoylphosphatidylglycerol, Lysophosphatidylglycerol, Phosphatidylinositol, 1,2-Di(2,4-octadecadienoyl)-sn-glycerol-3-phosphorylcholine and Phosphatidylcholines (e.g. Didecanoyl-L-phosphatidylcholine, Dilauroylphosphatidylcholine, DipalmitoylPhosphatidylcholine, Distearoylphosphatidylcholine), Lysophosphatidylcholine is of particular interest.

Cyclodextrins, for example:

β-Cyclodextrin, Dimethyl-β-Cyclodextrin, y-Cyclodextrin, Hydroxypropyl β-cyclodextrin, Methyl Cyclodextrin; especially Dimethyl-β-Cyclodextrin is of particular interest

Fusidic Acid derivatives, for example:

Sodium Taurodihydrofusidate, Sodium Glycodihydrofusidate, Sodium Phosphate-Dihydrofusidate; especially Sodium Taurodihydrofusidate is of particulare interest

Microspheres, for example:

Microspheres of Starch, Microspheres of Dextran, Micronspheres of Hyaluronic Acid Ester

Others:

Sodium salts of e.g. glycyrrhizic acid, capric acid, alkanes (e.g. azacycloalkanes), amines and amides (e.g. N-methyl-pyrrolidone, Azone), amino acids and modified amino acids compounds (e.g. acetyl-L-cysteine), polyols (e.g. propyleneglycol, hydrogels), sulfoxides (e.g. dimethylsulfoxide), terpenes (e.g. carvone), ammonium glycyrrizinate, hyluronic acid, isopropyl myristate, n-lauryl-beta-D-maltopyranoside, saponins, DL-octanonylcarnitine chloride, palmitoyl-DL-carnitine chloride, DL-stearoylcarnitine chloride, acylcarnitines, ethylenediaminedihydro-chloride, phosphate-dihydrofusidate, sodium CAP); especially n-lauryl-beta-D-maltopyranoside is of particular interest, alpha 1000 peptide, peptide MW<1000 comprising at least 6 mol % of aspartatic- and gGlutamic Acid, decomposed royal jelly, vitamin D₂, vitamin D₃, hydroxy-vitamin D₃, 1.25-dihydroxy-vitamin D₃, spirulina, proteoglycan, soyahydrolysate, lysin, lactic acid, di-fructose-anhydrid, xylitol Ca-(lactate), hydrolyzate of casein in particular a caseinoglycomacropeptide, negative ionization of CaCO₃, acetylsalicylic acid, vitamin K, creatin.

Other Specific Embodiments of the Invention

In order to test whether PTH is intact in a specific type of composition and/or whether the absorption characteristics are suitable, it is possible to substitute PTH with another peptide that is cheaper. Such peptides include lysozyme, aprotinin, desmopressien, vasopressien, insulin, GLP-1, GLP-1 fragment 7-37, calcitonin etc. Accordingly, the present invention also includes compositions wherein one or more of the above-mentioned peptides are incorporated (instead of PTH).

Preparation of a Pharmaceutical Composition According to the Invention

A pharmaceutical composition according to the invention may be prepared by use of any convenient method (see e.g. Remington's Pharmaceutical Handbook). A suitable method used by the present for the preparation of a composition according to the invention is described in the following Examples.

Other Aspects of the Invention

The invention also relates to a method for administering active substance to the small intestine or the colon, the method comprising administering to a patient a sufficient amount of a pharmaceutical composition according to the invention. Such a delivery system is typically designed so that it enables a relatively fast release, namely when the delivery system reaches GI target, i.e. the small intestine or the colon. The particulars and details given above under the main aspect of the invention applies mutatis mutandis to these further aspects.

### LEGENDS TO FIGURE

FIG. 1 shows schematically a first unit for use according to the invention. The unit comprises an inner core (in this example the core is cellulose sphere) surrounded by a layer containing the active substance. On top on this layer is the time controlled layer (here it is a swelling layer) that is coated with a water insoluble membrane. Finally, an enteric membrane is added.

In general, the layer containing the active substance constitute from about 0.5 to about 90% w/w such as, e.g., from about 1% w/w to about 80% w/w, from about 1.5% w/w to about 70% w/w, from about 2% w/w to about 60% w/w, from about 2% w/w to about 50% w/w of the first unit.

The time controlled layer normally constitutes from about 10% w/w to about 90% w/w such as, e.g., from about 20% w/w to about 90% w/w, from about 30% w/w to about 85% w/w of the first unit.

The water insoluble membrane normally constitutes from about 4% w/w to about 25% w/w of the first unit and the enteric membrane normally constitutes from about 2% w/w to about 25% w/w of the first unit.

FIG. 2 shows schematically a plasma concentration vs time profile in humans after oral administration of a PTH-containing composition according to the invention.

FIG. 3 shows schematically the change in PTH concentration followed by administration of a calcium-containing compound and a PTH, wherein the administration or composition employed ensures that calcium is rapidly released whereas the release of PTH is delayed. The initial effect of calcium lowers the plasma level of PTH and upon release of PTH, the plasma level of PTH increases significantly. The effect aimed at with respect to calcium is a decrease in PTH plasma level of about −50%, limits (−5%)-(−100%), and with respect to PTH once released is a change in PTH plasma level of about 650% (limits 10%-1200%).

In the Following is Listed Specific Embodiments of the Invention

1. A pharmaceutical composition for oral administration comprising PTH, wherein the in vitro release of PTH—when tested in a dissolution test of pharmacopoeia standard—is delayed with at least 2 hours and once the release starts, at least 90% w/w such as, e.g., at least 95% or at least 99% of all PTH contained in the composition is released within at the most 2 hours.

2. A pharmaceutical composition according to item 1, wherein—when tested in an in vitro dissolution test employing 0.1 N HCl equilibrated at 37° C. as the dissolution medium—at the most a bout 10% w/w such as, e.g., at the most about 7.5% w/w, at the most about 5% w/w, at the most about 2.5% w/w, at the most about 1% w/w of PTH contained in the composition is released 2 hours after start of the test.

3. A pharmaceutical composition according to item 1 or 2 for delivery of PTH to the small intestine and/or to the colon.

4. A pharmaceutical composition according to any of the preceding items for delivery of PTH to the jejunum.

5. A pharmaceutical composition according to item 4, wherein—when tested in an in vitro dissolution test employing a dissolution medium having a pH of about 6.8 and a temperature of about 37° C.—the following dissolution patterns of PTH are obtained (after start at pH 6.8):

| | |
|---|---|
| at 15 min. | approx. 20% w/w (limits 0-50% w/w) |
| at 30 min. | approx. 80% w/w (limits 25-100% w/w) |
| at 60 min. | approx. 100% w/w (limits 50-100% w/w) |

6. A pharmaceutical composition according to any of items 1-3 for delivery of PTH to ileum.

7. A pharmaceutical composition according to item 6, wherein—when tested in an in vitro dissolution test employing a dissolution medium having a pH of about 6.8 and a temperature of about 37° C.—the following dissolution patterns of PTH are obtained (after start at pH 6.8):

| | |
|---|---|
| at 2 hours 30 min | approx. 20% w/w (limits 0-50% w/w) |
| at 3 hours 30 min | approx. 80% w/w (limits 25-100% w/w) |
| at 4 hours 30 min | approx. 100% w/w (limits 50-100% w/w). |

8. A pharmaceutical composition according to any of items 1-3 for delivery of PTH to colon.

9. A pharmaceutical composition according to item 8, wherein—when tested in an in vitro dissolution test employing a dissolution medium having a pH of about 6.8 and a temperature of about 37° C.—the following dissolution patterns of PTH are obtained (after start at pH 6.8):

| | |
|---|---|
| at 4 hours | approx. 20% w/w (limits 0-50% w/w) |
| at 5 hours | approx. 80% w/w (limits 25-100% w/w) |
| at 6 hours | approx. 100% w/w (limits 50-100% w/w). |

10. A pharmaceutical composition according to any of the preceding items, wherein PTH is recombinant or of mammalian origin including human and is selected from full-length PTH (1-84) or its amino terminal fragment, PTH (e.g. PTH 1-34 etc).

11. A pharmaceutical composition according to any of the preceding items further comprising a calcium-containing compound.

12. A pharmaceutical composition according to item 11, wherein—when tested in an in vitro dissolution test employing 0.1 N HCl equilibrated at 37° C. as the dissolution medium—the following dissolution pattern of calcium is obtained:

| | |
|---|---|
| at 15 min. | approx. 20% w/w (limits 0-50% w/w) |
| at 30 min. | approx. 80% w/w (limits 25-100% w/w) |
| at 45 min. | approx. 100% w/w (limits 50-100% w/w). |

13. A pharmaceutical composition according to item 11 or 12, wherein the calcium-containing compound is selected from the group consisting of bisglycino calcium, calcium acetate, calcium carbonate, calcium chloride, calcium citrate, calcium citrate malate, calcium cornate, calcium fluoride, calcium glubionate, calcium gluconate, calcium glycerophosphate, calcium hydrogen phosphate, calcium hydroxyapatite, calcium lactate, calcium lactobionate, calcium lactogluconate, calcium phosphate, calcium pidolate, calcium stearate and tricalcium phosphate.

14. A pharmaceutical composition according to any of the preceding items further comprising a vitamin D (e.g. vitamin D₃).

15. A pharmaceutical composition according to any of the preceding items comprising a further therapeutically and/or prophylactically active substance that is effective in bone related disorders.

16. A pharmaceutical composition according to any of the preceding items further comprising an absorption enhancer.

17. A pharmaceutical composition according to any of the preceding items further comprising a PTH-stabilizing agent.

18. A pharmaceutical composition according to any of the preceding items in the form of a solid dosage form including tablets, capsules and sachets.

19. A pharmaceutical composition according to any of the preceding items in the form of a multiple unit dosage form comprising a multiplicity of the same or different pellets or granules.

20. A pharmaceutical composition according to any of the preceding item comprising one or more of a first type of unit, the first type of unit comprising PTH, and the first type of unit having a layered structure of at least

i) an inner core

ii) a time-controlled layer surrounding the inner core,

iii) a film coating applied on the time-controlled layer, wherein the film coating is substantially water insoluble but permeable to an aqueous medium, and

iv) an outer layer of an enteric coating.

21. A pharmaceutical composition according to item 20, wherein the release of the active substance from the unit—when tested in vitro as an average of at least three determinations—is not more than about 10% w/w at a first pH value below about 4.0, and at a second pH value of from about 5.0 to about 8.0 the active substance is released in such a manner that—after a lag time of from about 0.5 to about 8 hours in which first time period not more than about 10% w/w of the active substance is released—at least about 50% wlw of the active substance contained in the unit is released within a second time period of not more than about 2 hours.

22. A composition according to item 21, wherein the release of the active substance from the unit—when tested in vitro—is not more than about 7.5% w/w such as, e.g., not more than about 5% w/w, not more than about 2.5% w/w or not more than about 1% w/w at the first pH value below about 4.0.

23. A composition according to item 21, wherein the first pH value is below about 3.5, such as, e.g., below about 3.0, below about 2.5, below about 2.0, below about 1.5 or a pH value corresponding to that of 0.1 N HCl.

24. A composition according to any of item 20-23, wherein the lag time is from about 1.0 to about 7 hours such as, e.g., from about 1.5 to about 6 hours, from about 2.0 to about 5 hours or from about 2.5 to about 4.5 hours or from about 2.5 to about 4 hours.

25. A composition according to any of item 20-24, wherein—after said lag time—at least about 60% w/w such as, e.g., at least about 70% w/w, at least about 75% w/w, at least about 80% w/w, at least about 85% w/w, at least about 90% w/w, at least about 95% w/w or at least 99% w/w of the active substance contained in the unit is released within the second time period of not more than about 2 hours.

26. A composition according to any of items 21-25, wherein said second time period is not more than about 90 min such as, e.g., not more than about 60 min, not more than about 50 min, not more than about 45 min, not more than about 40 min, not more than about 35 min, not more than about 30 min, not more than about 25 min, not more than about 20 min, not more than about 15 min, not more than about 10 min of not more than about 5 min.

27. A pharmaceutical composition according to any of the preceding items provided with an enteric coating comprising an enteric polymer that has a pH cut off of at the most about 8.0 such as, e.g. in a range of from about 4.0 to about 7.5, in a range of from about 4.5 to about 7.0, from about 4.9 to about 6.9, from about 5.0 to about 6.5, from about 5.0 to about 6.3, from about 5.0 to about 6.0, from about 5.0 to about 5.9, from about 5.0 to about 5.7, from about 5.0 to about 5.6 or from about 5.0 to about 5.5.

28. A pharmaceutical composition according to any of items 20-27, wherein the core is selected from pharmaceutically acceptable beads, spheres, granules, granulates, and pellets.

29. A pharmaceutical composition according to item 28, wherein the lag time is controlled by the time it takes for the swellable layer to swell to such an extent that the film coating layer is disrupted or destructed.

30. A pharmaceutical composition according to any of items 20-29, wherein the lag time is controlled by the thickness and/or composition of the time-controlled layer.

31. A pharmaceutical composition according to any of items 20-30, wherein the lag time is further controlled by the thickness and/or composition of the film coating layer.

32. A pharmaceutical composition according to any of items 20-31, wherein the disruption or destruction of the film coating layer iii) is substantially independent of pH.

33. A pharmaceutical composition according to any of the preceding items in the form of a multiple unit composition.

34. A pharmaceutical composition according to any of items 1-32 in the form of a single unit composition.

35. A pharmaceutical composition according to any of the preceding items comprising i) a PTH, ii) a calcium containing compound, and iii) a vitamin D.

36. A pharmaceutical composition according to any of items 1-34 comprising i) PTH or a fragment, analog or derivative thereof, and ii) a vitamin D as active substances.

37. A pharmaceutical kit comprising a first and a second component, the first component comprising PTH and the second component comprising a calcium-containing compound, wherein the in vitro release of PTH—when tested in a dissolution test of pharmacopoeia standard—is delayed with at least 2 hours and once the release starts, at least 90% w/w such as, e.g., at least 95% or at least 99% of all PTH contained in the composition is released within at the most 2 hours.

38. A pharmaceutical kit according to item 37, wherein the first component comprising PTH comprises a composition as defined in any of items 1-36.

39. A pharmaceutical kit according to item 37 or 38, wherein the two components are contained in the same or different container.

40. A pharmaceutical kit according to any of items 37-39 further comprising instructions for use of the components.

41. A pharmaceutical kit according to any of items 37-40 further comprising a third component comprising a second dose of a calcium-containing compound and with instruction for substantially simultaneous oral intake of the first and the second component followed by oral intake of the third component after 2 hours or more such as, e.g., 3 hours or more, 4 hours or more, 5 hours or more, 6 hours or more, 7 hours or more, or 8 hours or more.

42. A pharmaceutical kit according to any of items 37-41 further comprising a vitamin D.

43. A pharmaceutical kit according to item 42, wherein vitamin D is included as one of the first or second components or as a separate component.

44. Use of a parathyroid hormone (PTH) in combination with a calcium-containing compound for the manufacture of a medicament for the treatment or prevention of bone-related diseases, wherein

i) an effective amount of a calcium-containing compound is administered to lower the plasma level of endogenous PTH,

ii) an effective amount of PTH is administered to obtain a peak concentration of PTH once the endogeneous PTH level is lowered.

45. Use according to item 44, wherein the calcium-containing compound and PTH is contained in the same or separate pharmaceutical compositions.

46. Use according to any of items 44-45, wherein the calcium containing compound is administered orally.

47. Use according to item 46, wherein PTH is administered at the most 4 hours later than the calcium containing compound.

48. Use according to any of items 44-47, wherein PTH is administered substantially simultaneous with the calcium containing compound.

49. Use according to any of items 44-48, wherein PTH and the calcium containing compound is contained in a composition as defined in any of items 1-36 or a kit as defined in any of items 37-43.

50. A method for administering active substances to the small intestine or colon, the method comprises administering to a patient a sufficient amount of a pharmaceutical composition defined in any of items 1-36, a kit as defined in any of items 37-43 or a medicament as defined in any of items 44-49.

51. A method for treatment or prevention of a bone related disorder including osteoporosis, the method comprising oral administration to a patient in need thereof a sufficient amount of PTH in a pharmaceutical composition as defined in any of items 1-36, a kit as defined in any of items 3743 or a medicament as defined in any of items 44-49.

The invention is further illustrated in the following non-limiting examples

Materials and Methods

In Vitro Dissolution Test Method

Apparatus: Ph.Eur./USP dissolution apparatus

Dissolution medium 1 (0 to 2 hours) acidic stage (up to pH 4.0)

Dissolution medium 2 (2 to 10 hours) buffer stage (pH 5.0 to 8.0)

| | |
|---|---|
| Time for medium change | 2 hours |
| Media Temperature | 37° C. ± 0.5° C. |

Agitation/flow rate/dip per minuteestablished by evaluating the specific formulation to be tested.

Detection systemestablished by evaluating the specific formulation to be tested

A number of units/capsules/tablets are tested. The test result is calculated by the use of a reference standard of the active substance. The test result is reported as the average of three or more—determinations.

A person skilled in the art is capable of defining appropriate testing method conditions for the specific pharmaceutical formulations described in present document.

### EXAMPLES

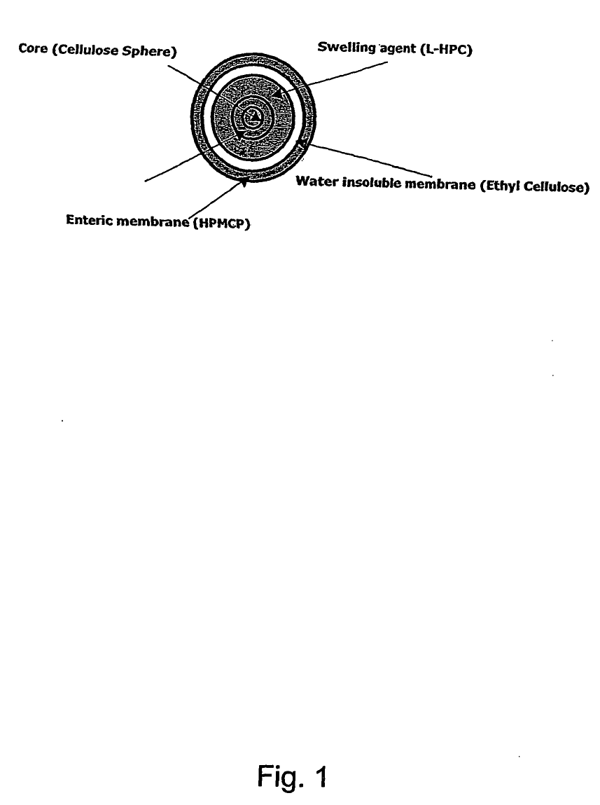

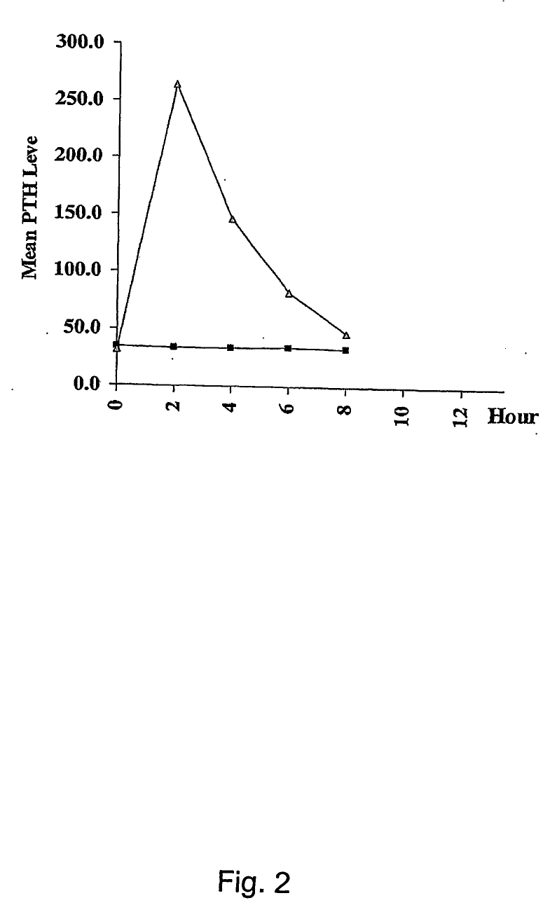

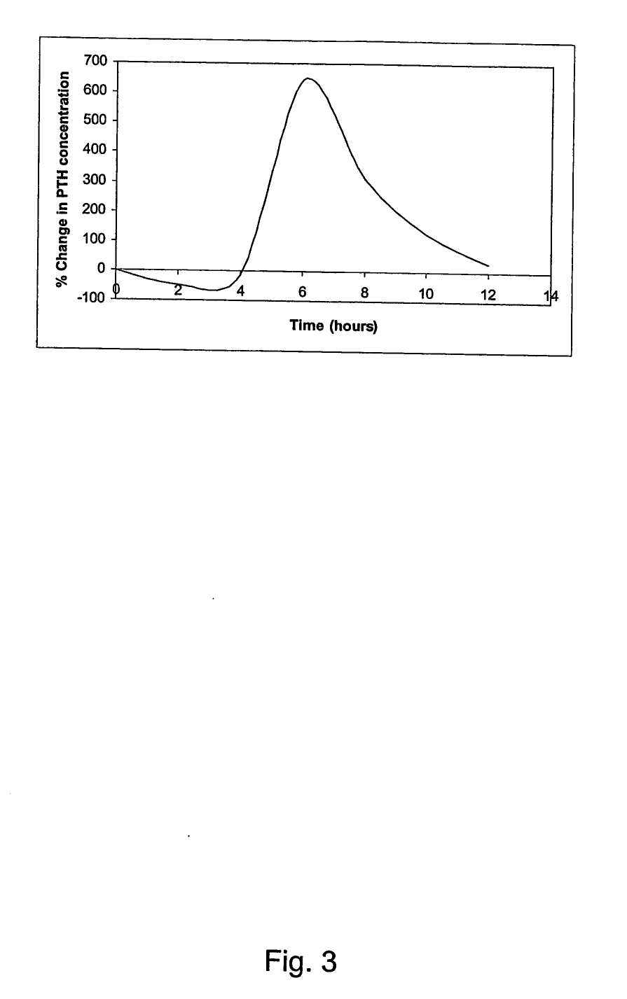

Some of the examples herein illustrate units with enteric membrane for jejunum and ileum delivery and units that have up to a 5 layer spherical structure for ileum and colon delivery, which contains a core, drug, swelling agent, water insoluble membrane and enteric membrane. After the system is administered via the oral route the enteric membrane prevents water from entering into the system as long as the system is in the stomach. When the system enters into a more alkaline environment the enteric membrane quickly dissolves and the pre-programmed lag time starts. The water penetrates through the insoluble but permeable membrane and starts hydrating the swelling agent. When stress by expansion of the hydrated swelling agent exceeds the tensile strength of the water insoluble membrane the disruption of the membrane occurs. Finally, the drug release is initiated (see FIG. 1). Drug release is triggered by membrane destruction and the time until the destruction creates the lag time for the release. The lag time can be controlled by the composition and/or thickness of the swelling agent and the water insoluble membrane but prolonging the lag time initiates larger variation on the lag time and decrease the release rate.

### Example 1

Preparation of Tablets Containing PTH for Intestinal Delivery (Jejunum)

The present example illustrates the preparation of tablets for intestinal delivery (Jejunum). The composition of the tablets is shown in table 1

TABLE 1

| Ingredients | Amount (g) |
|---|---|
| PTH (lyophilized PTH) | 120.0 |
| Trypsin inhibitor¹ | 600.0 |
| Sodium Laurylsulfate | 34 |
| Microcrystalline cellulose | 560.0 |
| Sodium carboxymethylcellulose | 560.0 |
| Polyvinylpyrrolidone 90 | 26 |
| Magnesium stearate | 10.0 |
| Talc | 90.0 |
| Total | 2000.0 |

1. Assuming effective concentration approx 0.5 mg/ml, Max volume of intestine is approx. 500 ml for 100 cm of intestine. Release as burst covering 20 cm of intestine i.e. effective dose needed is 0.5 mg/ml*500 ml*0.2 m=50 mg/dose

The ingredients were mixed and wet granulated in a high shear mixer and dried in a fluid-bed until the absolute water content was below 2%. The granulated powder was compressed into tablets by the use of a Fette exacta compression machine.

1.5 kg of these tablets was coated with a protection coat and an enteric coat in a Glatt GPCG 3 fluid-bed with a 1.2 mm spray nozzle and a spray pressure of 2.5 bars. The composition of the protection coat (8% w/w dry matter) and enteric coat (22.6% w/w dry matter) are shown in table 2 and 3.

TABLE 2

| Ingredients | Amount (g) |
|---|---|
| Hydroxypropyl methylcellulose E5 | 40.0 |
| Talc | 40.0 |
| Purified water | 920.0 |
| Total | 1000.0 |

TABLE 3

| Ingredients | Amount (g) |
|---|---|
| Eudragit L30D | 996.4 |
| Triethyl citrate | 29.89 |
| Talcum | 149.46 |
| Purified water | 944.25 |
| Total | 2120.0 |

In the coating process 2% w/w protection coat and 25% w/w enteric coat were applied. The amount of dry matter applied is calculated in percentage of the core weight.

The tablets were heated to 30° C. and throughout the coating process the product temperature was maintained substantially in the interval from 28 to 32° C. by adjustment of the liquid flow rate in the interval from 10 to 15 g/min. The inlet air temperature and the process airflow were kept at approximately 35° C. and 150 m³/h, respectively. After the application of the coatings the coated tablets were dried for 15 minutes. The mass of the tablets was approximately 200 mg.

Dissolution

For 2 hours in 0.1 N HCl practically no PTH was release (less than 0.5%). After 2 hours the pH in the dissolution media was changed to 6.8. 15 min after the pH changed 35% of the PTH dose was release, 75% of the PTH dose after 30 min and 100% of the PTH dose after 60 min.

### Example 2

Preparation of Tablets Containing PTH for Intestinal Delivery (Ileum)

The present example illustrates the preparation of tablets for intestinal delivery (ileum). The composition of the tablets is shown in table 4

TABLE 4

| Ingredients | Amount (g) |
|---|---|
| PTH (lyophilized PTH) | 120.0 |
| Amastatin² | 31.8 |
| Sodium deoxycholate³ | 720.0 |
| Microcrystalline cellulose | 500.1 |
| Sodium carboxymethylcellulose | 500.1 |
| Polyvinylpyrrolidone 90 | 28 |
| Magnesium stearate | 10.0 |
| Talc | 90.0 |
| Total | 2000.0 |

2. Assuming effective concentration approx 0.0265 mg/ml (50 μM), Max. volume of intestine is approx. 500 ml for 100 cm of intestine. Release as burst covering 20 cm of intestine i.e. effective dose needed is 0.0053 mg/ml*532 ml*0.2 m=0.56 mg/dose

3. Calculated as the 3% of the solid dosage form and not in the dissolved form.

Tablets were prepared and protection coat was applied as described in example 1.

1.5 kg of these tablets was coated with an enteric coat in a Glatt GPCG 3 fluid-bed with a 1.2 mm spray nozzle and a spray pressure of 2.5 bars. The composition of the coat (27% wlw dry matter) is shown in table 5.

TABLE 5

| Ingredients | Amount (g) |
|---|---|
| Eudragit FS30D | 2000.0 |
| Triethyl citrate | 30.0 |
| Talcum | 180.0 |
| Purified water | 790 |
| Total | 3000 |

In the coating process 25% w/w enteric coat was applied.

The tablets were heated to 20-25° C. and throughout the coating process the product temperature was maintained substantially in the interval from 20 to 25° C. by adjustment of the liquid flow rate in the interval from 10 to 15 g/min. The inlet air temperature and the process airflow were kept at approximately 35° C. and 100 m³/h, respectively. The coated tablets were dried for 30 minutes at 40° C. The mass of the tablets was approximately 200 mg.

Dissolution

For 2 hours in 0.1 N HCl practically no PTH was release (less than 0.5%). After 2 hours the pH in the dissolution media was changed to 6.8. Less than 5% of the PTH dose was release 4 hours after the pH changed. After total 6 hours the pH in the dissolution media was changed to 7.5. 30 min after the latest change 100% of the PTH dose was released.

### Example 3

Preparation of Cores Containing PTH for Colon Delivery

The present example illustrates the preparation of cores for colon delivery. The composition of the cores is shown in table 6.

The cores were prepared by the use of the extrusion/spheronization technique.

TABLE 6

| Ingredients | Amount (g) | |
|---|---|---|
| PTH (lyophilized PTH) | 400.0 | |
| Aprotinin⁴ | 250.0 | |
| EDTA | 1000.0 | |
| Microcrystalline cellulose | 337.5 | |
| Lactose monohydrate | 462.5 | |
| Sodium carboxymethylcellulose | 50.0 | |
| Purified water | 775 | g |

4. Assuming effective concentration approx 0.25 mg/ml, Max. volume of intestine is 500 ml for 100 cm of intestine. Release as burst covering 20 cm of intestine i.e. effective dose needed is 0.25 mg/ml*500 ml*0.2 m=25 mg/dose

The ingredients were mixed and wetted in a Fielder high shear mixer. The wetted mass was extruded in a Nica E 140 extruder with a 0.6 mm screen size. The extrudate was spheronized in a lab unit until the surface was smooth and the cores were spherical.

The cores were dried in a Glatt GPCG fluid-bed for approximately 30 minutes at 50° C. The dried cores were fractionated by screening through a lower screen of 600 μm and an upper screen of 800 μm.

### Example 4

Preparation of Cores With a Swelling Layer Using Suspension Coating

1 kg cores as obtained from example 3 were coated with a protection coat as described in example 1. Further the cores were coated with a swelling agent and an outer coat in a Glatt GPCG fluid-bed equipped with a rotary processor. The nozzle was placed in the lowest position. The distance from the wall to the nozzle point was 25 mm and the nozzle port size was 1.2 mm. The spray pressure was 2.5 bar and the rotations rate on the disk was 500 rpm. The product differential pressure was approximately 1.5 kPa. The composition of the suspension coat (25% w/w dry matter) and the outer coat (4.2% w/w dry matter) are shown in table 7 and 8.

TABLE 7

| Ingredients | Amount (g) |
|---|---|
| L-HPC LH-31 | 4472 |
| Hydroxypropyl cellulose L-/fine | 903 |
| Ethanol 99.9% | 16125 |
| Total | 21500 |

TABLE 8

| Ingredients | Amount (g) |
|---|---|
| Hydroxypropyl cellulose L-/fine | 63.0 |
| Ethanol 99.9% | 1437.0 |
| Total | 1500.0 |

In the coating process 400% w/w L-HPC and 1% w/w outer coat were applied.

The cores were heated to 25° C. and throughout the coating process the product temperature was kept at approximately 15° C. by adjustment of the liquid flow rate in the interval from 35 to 45 g/min. The humidified inlet air temperature and the process airflow were kept at approximately 25° C. and 100 m³/h, respectively. The coated cores were dried on trays for approximately 24 hours at 40° C. The dried cores were fractionated by screening through a lower screen of 710 μm and an upper screen of 1000 μm.

### Example 5

Preparation of Cores With an Aim of Obtaining a 3.5 Hours Lag Time

2 kg of cores as obtained from example 4 were coated with a water insoluble coat in a Glatt GPCG 3 fluid-bed. The composition of the water insoluble coat (10.9% w/w dry matter) is shown in table 9.

TABLE 9

| Ingredients | Amount (g) |
|---|---|
| Ethyl cellulose 20 | 563.0 |
| Polyethylene glycol 6000 | 197.0 |
| Colloidal silica dioxide | 113.0 |
| Ethanol 99.9% | 7127.0 |
| Total | 8000.0 |

In the coating process 42.2% w/w water insoluble coat was applied. The cores were heated to 30° C. and throughout the coating process the product temperature was maintained substantially in the interval from 28 to 31° C. by adjustment of the liquid flow rate in the interval from 10 to 20 g/min. The inlet air temperature and the process airflow were kept at approximately 35° C. and 100 m³/h, respectively. The coated cores were dried for 15 minutes. The coated cores were screened through a 1200 μm screen. Oversized material: <5% w/w.

### Example 6

Preparation of Cores for Colon Delivery

2 kg of cores as obtained from example 5 were coated with an enteric coat in a Glatt GPCG 3 fluid-bed, The composition of the enteric coat (7.5% w/w dry matter) is shown in table 10.

TABLE 10

| Ingredients | Amount (g) |
|---|---|
| Hydroxypropyl methylcellulose phthalate | 480.0 |
| Triethyl citrate | 24.0 |
| Colloidal silica dioxide | 96.0 |
| Purified water | 1110.0 |
| Ethanol 99.9% | 6290.0 |
| Total | 8000.0 |

In the coating process 29% w/w enteric coat was applied.

The cores were coated as described in example 5. The coated cores were screened through a 1200 μm screen. Oversized material: <5% w/w.

Dissolution

For 2 hours in 0.1 N HCl practically no PTH was released (less than 0.5%). After 2 hours the pH was changed to 6.8 and the dissolution followed for further 5 hours. The release of PTH was 5% of the dose after 3.5 hours in the dissolution media with the pH 6.8. After 4 hours 30% was released, after 4.5 hours 70% was released and after 5 hours 100% of the dose was released.

### Example 7

Preparation of Cores With PTH, a Swelling Agent Mixed With 4-MOAC and Chitosan-EDTA Conjugate Using Powder Layering

1 kg Cellulose Spheres with a particle size between 350-500 μm were coated with a PTH containing coat and a protection coat as described in example 1. The composition of the PTH coat (17.8% w/w dry matter) is shown in table 11.

TABLE 11

| Ingredients | Amount (g) |
|---|---|
| PTH (lyophilized PTH) | 250.0 |
| Hydroxypropyl methylcellulose E5 | 63.9 |
| Talc | 42.6 |
| Purified water | 1643.5 |
| Total | 2000.0 |

In the coating process 35.6% w/w PTH coat and 2% w/w protection coat were applied.

Further the cores were coated with a pre-sieved mixture of 600 g 4-MOAC, 540 g Chitosan-EDTA and 3.74 kg L-HPC LH-31 by layering while simultaneously spraying a binder solution in a Glaft GPCG fluid-bed equipped with a rotary processor (see example 4). The composition of the binder solution (5% w/w dry matter) is given in Table 12.

TABLE 12

| Ingredients | Amount (g) |
|---|---|
| Hydroxypropyl cellulose L-/fine | 100.0 |
| Ethanol 99.9% | 1900.0 |
| Total | 2000.0 |

In the coating process 10% w/w 4-MOAC, 9% w/w Chitosan-EDTA, 374% w/w L-HPC and 1% w/w outer coat were applied (based on the weight of the core). The binder solution was also used as outer coat.

The cores were heated to 25° C. and throughout the coating process the product temperature was kept at approximately 25° C. by adjustment of the liquid flow rate in the interval from 35 to 45 g/min. The inlet air temperature and the process airflow were kept at approximately 35° C. and 100 m³/h, respectively. The coated cores were dried to water content below 2% w/w on trays at 30° C. The dried cores were fractionated by screening through a lower screen of 750 μm and an upper screen of 1000 μm. The content of PTH was at least 95% w/w.

### Example 8

Preparation of Cores Containing PTH, 4-MOAC and Chitosan-EDTA Conjugate for Colon Delivery

2 kg of cores from example 7 were coated with a water insoluble coat (applying 40% w/w) and an enteric coat (applying 20% w/w) in a Glatt GPCG 3 fluid-bed as described in Examples 5 and 6. The coated cores were screened through a 1.2 mm screen. Oversized material: <5%.

Dissolution

After 2 hours in 0.1 N HCl practically no PTH was released (less than 0.5%). The pH was changed to pH 6.8 and the dissolution followed for further 5 hours. The release of PTH was 5% of the dose after 3.5 hours in the dissolution media with the pH at 6.8. After 4 hours 25% was released, after 4.5 hours 60% was released and after 5 hours 100% of the dose was released.

### Example 9

Oral Preparation of Parathyroid Hormone (PTH) Modified Release Composition Made in the Form of Capsules Containing Multiple Units

A once daily oral PTH product (10 mg) to take in addition to a supplement of 1000-1500 mg Calcium and 400-12001U or higher doses of Vitamin D₃ (e.g. 1-3 Calcichew-D₃ tablets). The modified release PTH product was prepared by filling cores as obtained from Example 6 or 8 into hard gelatine capsules. The mass of the capsules was approximately 400 mg. The Calcium supplement should be taken with a meal during daytime and the PTH product should be taken in the evening either in connection with the evening meal or just before bed time. The release of PTH will be delayed for approximately 3.5 to 6 hours (depending on gastric pH and gastric emptying) and thereby will not interfere with the beneficial effect obtained from the calcium supplement. Any possible adverse effects of this PTH treatment will occur while the patient is asleep.

### Example 10

Preparation of Granulates Containing Calcium Carbonate and Vitamin D₃

Granulates were prepared as described in example 1. The composition is shown in Table 13.

TABLE 13

| Ingredients | Amount (g) |
|---|---|
| Cholecalciferol | 50 |
| Microcrystalline cellulose | 375 |
| Sodium carboxymethylcellulose | 50 |
| Calcium carbonate | 2000 |
| Polyvinylpyrrolidone 90 | 25 |

### Example 11

Preparation of a Combinations Product Containing Calcium Carbonate, Vitamin D₃ and PTH in the Form of Sachets Containing Granules and Multiple Units

Once daily product containing 500 mg calcium, 400-1200 IU or higher doses of Vitamin D₃ and 10 mg PTH. The product was prepared by mixing the granules from example 10 and cores obtained from Examples 6 or 8 into sachets. The mass of the sachets was approximately 1900 mg.

This product should be taken in the evening. The release of PTH will be delayed for approximately 3.5 to 6 hours (depending on gastric pH and gastric emptying) and thereby will not interfere with the beneficial effect obtained from the calcium supplement. Any possible adverse effects of this PTH treatment will occur while the patient is asleep. The patient should be advised to take an additional supplement of Calcium and Vitamin D₃ (e.g. 1-2 Calcichew-D₃ tablets) during daytime.

1. A pharmaceutical composition for oral administration comprising PTH, wherein the in vitro release of PTH—when tested in a dissolution test of pharmacopoeia standard—is delayed with at least 2 hours and once the release starts, at least 90% w/w of all PTH contained in the composition is released within at the most 2hours.

2. A pharmaceutical composition according to claim 1, wherein—when tested in an in vitro dissolution test employing 0.1 N HCl equilibrated at 37° C. as the dissolution medium—at the most about 10% w/w contained in the composition is released 2 hours after start of the test.

3. A pharmaceutical composition according to claim 1 for delivery of PTH to the small intestine and/or to the colon.

4. A pharmaceutical composition according to claim 1 for delivery of PTH to the jejunum.

5. A pharmaceutical composition according to claim 4, wherein—when tested in an in vitro dissolution test employing a dissolution medium having a pH of about 6.8 and a temperature of about 37° C.—the following dissolution patterns of PTH are obtained (after start at pH 6.8):

| | |
|---|---|
| at 15 min. | approx. 20% w/w (limits 0-50% w/w) |
| at 30 min. | approx. 80% w/w (limits 25-100% w/w) |
| at 60 min. | approx. 100% w/w (limits 50-100% w/w) |

6. A pharmaceutical composition according to claim 1 for delivery of PTH to ileum.

7. A pharmaceutical composition according to claim 6, wherein—when tested in an in vitro dissolution test employing a dissolution medium having a pH of about 6.8 and a temperature of about 37° C.—the following dissolution patterns of PTH are obtained (after start at pH 6.8):

| | |
|---|---|
| at 2 hours 30 min | approx. 20% w/w (limits 0-50% w/w) |
| at 3 hours 30 min | approx. 80% w/w (limits 25-100% w/w) |
| at 4 hours 30 min | approx. 100% w/w (limits 50-100% w/w). |

8. A pharmaceutical composition according to claim 1 for delivery of PTH to colon.

9. A pharmaceutical composition according to claim 8, wherein—when tested in an in vitro dissolution test employing a dissolution medium having a pH of about 6.8 and a temperature of about 37° C.—the following dissolution patterns of PTH are obtained (after start at pH 6.8):

| | |
|---|---|
| at 4 hours | approx. 20% w/w (limits 0-50% w/w) |
| at 5 hours | approx. 80% w/w (limits 25-100% w/w) |
| at 6 hours | approx. 100% w/w (limits 50-100% w/w). |

10. A pharmaceutical composition according to claim 1, wherein PTH is recombinant or of mammalian origin including human and is selected from full-length PTH (1-84) or its amino terminal fragment, PTH (e.g. PTH 1-34 etc).

11. A pharmaceutical composition according to claim 1 further comprising a calcium-containing compound.

12. A pharmaceutical composition according to claim 11, wherein—when tested in an in vitro dissolution test employing 0.1 N HCl equilibrated at 37° C. as the dissolution medium—the following dissolution pattern of calcium is obtained:

| | |
|---|---|
| at 15 min. | approx. 20% w/w (limits 0-50% w/w) |
| at 30 min. | approx. 80% w/w (limits 25-100% w/w) |
| at 45 min. | approx. 100% w/w (limits 50-100% w/w). |

13. A pharmaceutical composition according to claim 11, wherein the calcium-containing compound is selected from the group consisting of bisglycino calcium, calcium acetate, calcium carbonate, calcium chloride, calcium citrate, calcium citrate malate, calcium cornate, calcium fluoride, calcium glubionate, calcium gluconate, calcium glycerophosphate, calcium hydrogen phosphate, calcium hydroxyapatite, calcium lactate, calcium lactobionate, calcium lactogluconate, calcium phosphate, calcium pidolate, calcium stearate and tricalcium phosphate.

14. A pharmaceutical composition according to claim 1 further comprising a vitamin D (e.g. vitamin D₃).

15. A pharmaceutical composition according to claim 1 comprising a further therapeutically and/or prophylactically active substance that is effective in bone related disorders.

16. A pharmaceutical composition according to claim 1 further comprising an absorption enhancer.

17. A pharmaceutical composition according to claim 1 further comprising a PTH-stabilizing agent.

18. A pharmaceutical composition according to claim 1 in the form of a solid dosage form including tablets, capsules and sachets.

19. A pharmaceutical composition according to claim 1 in the form of a multiple unit dosage form comprising a multiplicity of the same or different pellets or granules.

20. A pharmaceutical composition according to claim 1 comprising one or more of a first type of unit, the first type of unit comprising PTH, and the first type of unit having a layered structure of at least
i) an inner core
ii) a time-controlled layer surrounding the inner core,
iii) a film coating applied on the time-controlled layer, wherein the film coating is substantially water insoluble but permeable to an aqueous medium, and
iv) an outer layer of an enteric coating.

21. A pharmaceutical composition according to claim 20, wherein the release of the active substance from the unit—when tested in vitro as an average of at least three determinations—is not more than about 10% w/w at a first pH value below about 4.0, and at a second pH value of from about 5.0 to about 8.0 the active substance is released in such a manner that—after a lag time of from about 0.5 to about 8 hours in which first time period not more than about 10% w/w of the active substance is released—at least about 50% w/w of the active substance contained in the unit is released within a second time period of not more than about 2 hours.

22. A composition according to claim 21, wherein the release of the active substance from the unit—when tested in vitro—is not more than about 7.5% w/w at the first pH value below about 4.0.

23. A composition according to claim 21, wherein the first pH value is below about 3.5 1.5 or a pH value corresponding to that of 0.1 N HCl.

24. A composition according to claim 20, wherein the lag time is from about 1.0 to about 7 hours.

25. A composition according to claim 20, wherein—after said lag time—at least about 60% w/w of the active substance contained in the unit is released within the second time period of not more than about 2 hours.

26. A composition according to claim 21, wherein said second time period is not more than about 90 min.

27. A pharmaceutical composition according to claim 1 provided with an enteric coating comprising an enteric polymer that has a pH cut off of at the most about 8.0.

28. A pharmaceutical composition according to claim 20, wherein the core is selected from pharmaceutically acceptable beads, spheres, granules, granulates, and pellets.

29. A pharmaceutical composition according to claim 28, wherein the lag time is controlled by the time it takes for the swellable layer to swell to such an extent that the film coating layer is disrupted or destructed.

30. A pharmaceutical composition according to claim 20, wherein the lag time is controlled by the thickness and/or composition of the time-controlled layer.

31. A pharmaceutical composition according to claim 20, wherein the lag time is further controlled by the thickness and/or composition of the film coating layer.

32. A pharmaceutical composition according to claim 20, wherein the disruption or destruction of the film coating layer iii) is substantially independent of pH.

33. A pharmaceutical composition according to claim 1 in the form of a multiple unit composition.

34. A pharmaceutical composition according to claim 1 in the form of a single unit composition.

35. A pharmaceutical composition according to claim 1 comprising i) a PTH, ii) a calcium containing compound, and iii) a vitamin D.

36. A pharmaceutical composition according to claim 1 comprising i) PTH or a fragment, analog or derivative thereof, and ii) a vitamin D as active substances.

37. A pharmaceutical kit comprising a first and a second component, the first component comprising PTH and the second component comprising a calcium-containing compound, wherein the in vitro release of PTH—when tested in a dissolution test of pharmacopoeia standard—is delayed with at least 2 hours and once the release starts, at least 90% w/w of all PTH contained in the composition is released within at the most 2 hours.

38. A pharmaceutical kit according to claim 37, wherein the first component comprising PTH comprises a composition as defined in claim 1.

39. A pharmaceutical kit according to claim 37, wherein the two components are contained in the same or different container.

40. A pharmaceutical kit according to claim 37 further comprising instructions for use of the components.

41. A pharmaceutical kit according to claim 37 further comprising a third component comprising a second dose of a calcium-containing compound and with instruction for substantially simultaneous oral intake of the first and the second component followed by oral intake of the third component after 2 hours or more.

42. A pharmaceutical kit according to claim 37 further comprising a vitamin D.

43. A pharmaceutical kit according to claim 42, wherein vitamin D is included as one of the first or second components or as a separate component.

44-49. (canceled)

50. A method for administering active substances to the small intestine or colon, the method comprises administering to a patient a sufficient amount of a pharmaceutical composition defined in claim 1.

51. A method for treatment or prevention of a bone related disorder including osteoporosis, the method comprising oral administration to a patient in need thereof a sufficient amount of PTH in a pharmaceutical composition as defined in claim 1.
